(19) United States
(12) Patent Application Publication
(10) Pub. No.: US 2008/0318891 A1
(43) Pub. Date: Dec. 25, 2008

(54) Antisense oligonucleotides against thymidylate synthase

(76) Inventors: Donald James Koropatnick, London (CA); Nicholas Mark Dean, Encinitas, CA (US); Mark D. Vincent, London (CA)

Correspondence Address: Gary M. Nath;THE NATH LAW GROUP, 112 South West Street, Alexandria, VA, 22314, US

(21) Appl. No.: 11/987,568

(22) Filed: Nov. 30, 2007

Related U.S. Application Data
(63) Continuation-in-part of application No. 10/597,409.
Application No. 10/597,409 is a continuation-in-part of application No. 11/171,435, filed on Jul. 1, 2005, now abandoned.
Application No. 11/171,435 is a continuation of application No. 09/509,418, filed on Jul. 11, 2000, now abandoned.

(51) Int. Cl. A61K 31/711; C07H 21/04

(52) U.S. Cl. 514/44; 536/24.5

(57) ABSTRACT

Antisense oligonucleotides targeted to sequences in thymidylate synthase (TS) mRNA. In particular the invention relates to antisense oligonucleotides targeted to sequences in the 3′ end of TS mRNA, which are both cytostatic on their own when administered to human tumour cell lines, and which also enhance the toxicity of anticancer drugs. The invention further relates to a combination product comprising an antisense oligonucleotide in combination with an anticancer agent such as Tomudex or pemetrexed and to the use of such a combination product in the treatment of cancer.

### FIELD OF THE INVENTION

The present invention pertains to the field of cancer therapeutics and in particular to antisense oligonucleotides against thymidylate synthase for the treatment of cancer.

### BACKGROUND OF THE INVENTION

Thymidylate synthase (TS) (EC 2.1.1.45) catalyses the conversion of deoxyuridylate to thymidylate, and is a housekeeping enzyme essential for the only intracellular de novo synthesis of thymidylate (Danenberg, 1977, Biochim. Biophys. Acta 473: 73-92). TS gene expression is tightly regulated with respect to cell proliferation state (Maley and Maley, 1960, J. Biol. Chem. 235: 2968-2970; Lochsin et al., 1979, Proc. Natl. Acad. Sci. USA 76: 750-754). As such, the TS gene is part of a group of genes whose expression is elevated at the G₁/S cell cycle boundary, and it has been suggested that transcription of several S-phase genes (including dihydrofolate reductase and thymidine kinase) is controlled in part by the E2F family of transcription factors (Farnham et al., 1993, Biochim. Biophys. Acta Rev. Cancer 1155: 125-131; Mudrak et al., 1994, Mol. Cell. Biol. 14: 1886-1892). In fact, transfection of active E2F1 genes into mouse cells induces expression of TS and other S-phase and cell cycle-regulated genes (De Gregori et al., 1995, Mol. Cell. Biol. 15: 4215-4224). As cells progress through the cell cycle from Gₒ through S phase, TS mRNA levels increase approximately 20-fold and TS enzyme activity increases about 10-fold (Navalgund et al., 1980, J. Biol. Chem. 255: 7386-7390). However, TS gene transcription rate is upregulated only 2 to 4 times, suggesting that post-transcriptional events play a major role in TS regulation (Ayusawa et al., 1986, J. Mol. Biol. 190: 559-567; Jenh et al., 1985, Mol. Cell. Biol. 5: 2527-2532; Johnson, 1994, J. Cell. Biochem. 54: 387-392). Differences in TS mRNA stability are not likely to be critical in regulation, as TS in RNA half-life is about 8 hours in both resting and growing rodent cells (Jenh et al., 1985, ibid). On the other hand, TS mRNA translation appears to be regulated by the TS protein itself, which specifically interacts at two sites within its own mRNA to inhibit protein production (Chu et al., 1991, Proc. Natl. Acad. Sci. USA 88: 8977-8981; Chu et al., 1993, Proc. Natl. Acad. Sci. USA 90: 517-521; Chu et al., 1994, Mol. Cell. Biol. 14: 207-213; Voeller et al., 1995, Nucleic Acids Res. 23: 869-875). Translation of other mRNAs (including c-myc mRNA) may also be regulated by interactions with TS protein (Chu et al., 1995, Mol. Cell. Biol. 15: 179-185).

Because of its role in DNA precursor synthesis, TS has been identified as a potential target for cancer chemotherapeutic agents (Hardy et al., 1987, Science 235: 448-455). High TS levels have been correlated with poor prognosis in patients with ovarian cancer (Suzuki et al., 1994, Oncology 51: 334-338), rectal cancer (Johnston et al., 1994, J. Cell. Biochem. 54: 387-392) childhood acute non-lymphoblastic leukaemia (Volm et al., 1994, Anticancer Res. 14: 1271-1276), and non-small cell lung carcinoma (Volm and Mattern, 1992, Anticancer Res. 12: 2293-2296). However, its prognostic value is not high in all tumour types (Peters et al., 1986, Cancer Res. 46: 20-28; Peters et al., 1994, Eur. J. Cancer 30A: 1408-1411).

Two types of TS inhibitors have been developed: (a) nucleotide analogues (including 5-FU, its riboside, and deoxyriboside derivatives) which must be activated to 5-fluorodeoxyuridylate (FdUMP) within cells to be effective (Heidelberger et al., 1983, Adv. Enzymol. 54: 58-119) and (b) 5,10-CH₂FH₄ (antifolate) analogues, including N-10-propargyl-5,8-dideazafolate (CB3717) (Calvert et al., 1986, J. Clin. Oncol. 4: 1245-1252) and Tomudex (ZD1694; N-[5-(N-[3,4-dihydro-2-methyl-4-oxoquinazolin-6-ylmethyl]-N-methylamino)-2-thenoyl]-L-glutamic acid) (Jackman et al., 1991, Adv. Enzyme Regul. 31: 13-27; Jackman et al., 1991, Cancel Res. 51: 5579-5586). Although Tomudex and 5-FU inhibit TS and have potent cytotoxic and antitumour activity (Heidelberger et al., 1983, ibid; Keyomarsi et al., 1993, J. Biol. Chem. 268: 15142-15149), they have an unusual biochemical effect. When human cancer cell lines are treated with 5-FU or Tomudex, TS levels increase rapidly, perhaps as a result of the release of translational inhibition by the TS protein (Keyomarsi et al., 1993, ibid; Chu et al., 1990, Cancer Res. 50: 5834-5840; Chu et al., 1993, Mol. Pharm. 43: 527-533).

It has been speculated that the release of translational inhibition that accompanies binding and inactivation of TS by chemotherapeutic agents (including Tomudex and 5-FU) might be prevented by treating cells with agents that could replace the specific interaction between TS mRNA and TS protein, and inhibit translation (Keyomarsi et al., 1993, ibid) but no such agents were described. In another speculative article it was hypothesised that antisense nucleic acids designed to both reduce the ability of TS mRNA to direct protein production, and to interact with the TS protein binding site, may be useful in complementing the effectiveness of drugs targeted against TS (Rapaport et al., 1992, Proc. Natl. Acad. Sci. USA 89: 8577-8580).

Chemotherapeutics that inhibit TS, such as 5-fluorouracil (5-FU) and its variants, have become integral drugs in standard treatments for colorectal cancer (Papamichael, 1999, Oncologist. 4:478-487). Raltitrexed (Tomudex®) and pemetrexed (Alimta®) are other TS inhibiting chemotherapeutics with a potential role in a range of cancers including mesothelioma. Although reasonably successful in clinical use, these drugs suffer from problems of dose-limiting toxicity and outgrowth of resistant cells, motivating the continued search for alternative treatments.

In previous patent applications, UK 9720107.3 and UK 9722012.3, specific down-regulation of the expression of TS in human breast cancer (MCF-7) cells in two ways was described. First, cells were transiently and stably transfected with vectors expressing antisense RNA molecules directed to hybridise to three different regions of the TS mRNA. Targeted sequences were: (1) sequences participating in the formation of a putative stem-loop structure surrounding the translation start site, and immediately adjacent and 3′ to that site (these sequences also participate in binding TS protein to modulate translation), (2) the exon1/exon2 boundary, and (3) the 3′ end of the mature cytoplasmic mRNA. Antisense TS RNA was expressed from these vectors (as assessed by Northern blot analysis and a novel modification of the run-on transcription assay to measure antisense transcription against background constitutive TS gene expression) (Koropatnick et al., 1997, BioTechniques 22: 64-66). Second, cells were transiently transfected with single-stranded oligodeoxynucleotides targeted to hybridise to: (a) the translation start site and sequences surrounding it, (b) a sequence proximal to the translation start site and participating in the putative stem-loop structure, and (c) the translation stop site near the 3′ end of the mature cytoplasmic RNA.

This background information is provided for the purpose of making known information believed by the applicant to be of possible relevance to the present invention. No admission is necessarily intended, nor should be construed, that any, of the preceding information constitutes prior art against the present invention.

### SUMMARY OF THE INVENTION

An object of the present invention is to provide antisense oligonucleotides against thymidylate synthase.

The present invention is based on the identification of the ability of an antisense oligonucleotide, oligo 83 [SEQ ID NO:1], complementary to a sequence in the TS mRNA 3′ untranslated region, to down-regulate the level of TS mRNA and protein, inhibited cell proliferation and enhanced the cytotoxicity of TS-directed chemotherapy drugs.

In one aspect of the invention, there is provided a composition comprising an antisense oligonucleotide of between 10 and 100 nucleotides in length and comprising at least 10 consecutive nucleotides of a sequence according to SEQ ID NO:1 or SEQ ID NO:2 and a pharmaceutically acceptable carrier or diluent.

In accordance with another aspect of the invention, there is provided a combination product comprising an antisense oligonucleotide of between 10 and 100 nucleotides in length and comprising at least 10 consecutive nucleotides of a sequence according to SEQ ID NO:1 or SEQ ID NO:2 in combination with an anticancer agent, wherein the antisense oligonucleotide hybridizes to a 3′ untranslated region of a mammalian thymidylate synthase nucleic acid and inhibits thymidylate synthase expression in mammalian cells.

In accordance with another aspect of the invention, there is provided a method for the treatment of cancer comprising administering to a human an effective amount of an antisense oligonucleotide of between 10 and 100 nucleotides in length and comprising at least 10 consecutive nucleotides of a sequence according to SEQ ID NO:1 or SEQ ID NO:2 and a pharmaceutically acceptable carrier or diluent.

In accordance with another aspect of the invention, there is provided a method for the treatment of cancer comprising administering to a human an effective amount of a combination product comprising an antisense oligonucleotide of between 10 and 100 nucleotides in length and comprising at least 10 consecutive nucleotides of a sequence according to SEQ ID NO:1 or SEQ ID NO:2 in combination with an anticancer agent, wherein the antisense oligonucleotide hybridizes to a 3′ untranslated region of a mammalian thymidylate synthase nucleic acid and inhibits thymidylate synthase expression in mammalian cells.

### BRIEF DESCRIPTION OF THE FIGURES

FIG. 1 shows that MCF-7 cell growth is inhibited by transfection with antisense TS oligo 86 (targeted to the translation stop site), but is enhanced by transfection with antisense TS oligos 90 or 92 (targeted to sequences at or near the translation start site). Cells were transiently-transfected with 0.5 or 1.0 μM antisense TS oligonucleotides in Lipofectin as described. Cell numbers were measured by Coulter counter in triplicate flasks after 4 days of growth. Control cells were treated with Lipofectin without oligonucleotides. Cell growth is expressed as a percentage of growth of control cells. *: Significantly higher than control (p<0.05, one way analysis of variants). **: Significantly lower than control (p<0.05, one way analysis of variants).

FIG. 2 shows that HeLa cell growth is inhibited by transfection with antisense TS oligo 86 (targeted to the translation stop site), but is enhanced after transfection with antisense TS oligo 91 (targeted to the translation start site). HeLa cells were transfected with 0.05 or 0.10 μM antisense TS oligonucleotides in Lipofectin (10 μg/ml) for 4 hours as described. Note that oligo concentrations are considerably lower than those used for MCF-7 cells. The efficiency of Lipofectin-mediated DNA transfection of HeLa cells is greater than for MCF-7 cells. Lipofectin was removed, the cells were trypsinised, and 25,000 viable cells per flask were plated in tissue culture flasks. Cell numbers were measured by Coulter counter in triplicate flasks after 4, 7 and 8 days of growth. Control cells were treated with Lipofectin without oligonucleotides. Cell growth is expressed as a percentage of growth of control cells. *: Significantly higher than control (p<0.05, Student's t-test). **: Significantly lower than control (Student's t-test).

FIG. 3 shows HeLa cell growth is inhibited by transfection with antisense TS oligo 83 (targeted to a 3′ untranslated sequence downstream of the translation stop site), but is not affected by transfection with antisense TS oligo 81 (targeted to the translation start site). The experimental protocol was as described in the legend to FIG. 2.

FIG. 4 shows that transient transfection of HeLa cells with oligo 86 (targeted to the TS translation stop site) enhances sensitivity to Tomudex and that oligo 91 (targeted to the TS translation start site) reduces sensitivity to Tomudex. HeLa cells were transfected with 0.05 and 0.10 μM antisense TS oligonucleotides and plated in flasks at low density, as described for FIG. 2. Tomudex (0-8 nM) was added (triplicate flasks for each Tomudex concentration) and the cells allowed to proliferate for 7 days. Cell numbers were measured by Coulter counting at that time. Survival is plotted as a percentage of growth in cells transfected with oligonucleotide, but untreated with Tomudex. Therefore, these data reveal inhibition or enhancement of Tomudex killing independent of growth inhibition or enhancement induced by oligonucleotides in the absence of Tomudex. The mean of three values is plotted. Error bars were smaller than the size of the symbol in each case. *: Significantly different from control (p<0.05, Student's t-test).

FIG. 5 shows that transient transfection of HeLa cells with oligo 83 (targeted to a sequence in the 3′ untranslated region of TS mRNA) enhances sensitivity to Tomudex whereas oligo 81 (targeted to a 3′ sequence downstream of that targeted by oligo 81) has no effect on Tomudex sensitivity. HeLa cells were transfected with 0.10 μM antisense TS oligonucleotides and plated in flasks at low density, as described for FIG. 2. Tomudex (0-10 nM) was added (triplicate flasks for each Tomudex concentration) and the cells allowed to proliferate for 4 (lays. Cell numbers were measured by Coulter counting at that time. Survival is plotted as a percentage of growth in cells transfected with oligonucleotide, but untreated with Tomudex. Therefore, these data reveal enhancement of Tomudex killing independent of growth inhibition induced by oligonucleotides in the absence of Tomudex. The mean of three values is plotted. Error bars were smaller than the size of the symbol in each case. *: Significantly different from control (p<0.05, Student's t-test).

FIG. 6 shows that antisense TS oligo 91, but not oligo 86, stimulates TS gene transcription in human HeLa cells. The same HeLa cells for which data are presented in FIG. 2 were assessed for run-on transcription of TS, glyceraldehyde phosphate dehydrogenase (GAPDH), and 18S rRNA genes. Briefly, cells were transfected with 0.05 and 0.10 μM antisense TS oligonucleotides in Lipofectin (10 μg/ml), or with Lipofectin alone (LFA control) for 4 hours as described. Lipofectin was removed and cells were trypsinised and replated in tissue culture flasks. Four days after transfection, nuclei were isolated from approximately 5×10⁶ cells for each treatment and initiated TS, GAPDH, and 18S rRNA transcripts allowed to incorporate [³²P]-CTP for 30 minutes. Alcohol-precipitable radiolabeled RNA was hybridised for 48 hours to unlabeled TS, GAPDH, and 18S rRNA cDNA immobilised in triplicate dots on nylon membrane as described. Relative transcription rate is presented as:

$$Relative transcription rate = \frac{( hybridisation signal from gene of interest )}{( hybridisation signal for GAPDH or 18 S rRNA genes )}$$

FIG. 7 shows the sequence of human mRNA for thymidylate synthase (EC 2.1.1.45), bases 1 to 1536.

FIG. 8 shows that antisense TS ODN 83 inhibits HeLa cell proliferation. HeLa cells transfected with 50 nM antisense TS ODN 83 (•) or 50 nM scrambled control ODN 32 (∘) were assessed for cell proliferation at 1, 2, 5, and 6 days following transfection. Data points indicate the average of two measurements, and are representative of qualitatively similar results obtained in 16 independent experiments.

FIG. 9 shows that antisense TS ODN 83 suppresses HeLa cell growth after transfection, followed by recovery to control proliferation rate after 48 hours. HeLa cells were transfected with 50 nM antisense TS ODN 83 or 50 nM scrambled control ODN 32 as described in the legend to FIG. 1. Values derived from cells transfected with ODN 32 were normalized to 100%, and each bar indicates the percent of that value measured following treatment with ODN 83 (mean±SE of 4 independent experiments). Asterisks (*) indicate significant differences (p<0.02, Student t-test).

FIGS. 10 & 11 show that treatment of HeLa cells with ODN 83 leads to decreased TS mRNA levels. (FIG. 10) HeLa cells were transfected with ODN 83 or scrambled control ODN 32, or treated with Lipofectamine alone. Cells were harvested at 1, 2, and 4 days post-transfection and total cellular RNA isolated, reverse-transcribed, and TS and GAPDH cDNA amplified by 24 PCR cycles in the same reaction vessel. TS (208 bp) and GAPDH (752 bp) RT/PCR products were confirmed by Southern blotting and hybridisation to specific radioactively-labeled probes.

(FIG. 11) TS:GAPDH ratio of RT/PCR products from RNA isolated from HeLa cells 1 day after transfection with ODN 83 or ODN 32. Twenty-four, 25, 26, or 27 cycles of PCR amplification were carried out, revealing the same reduction in TS:GAPDH ratio after transfection with ODN 83.

FIG. 12 shows that TS protein levels (inferred by measurements of 5-FdUMP binding) are diminished by antisense TS ODN 83 but not scrambled control ODN 32. 5-FdUMP binding was measured in cells transfected with ODN 83 (hatched bars) or ODN 32 (open bars) at different times following transfection. (A) Results are plotted as a percent of 5-FdUMP binding in cells transfected with control ODN 32±SE (n=5). The values for ODN 32 (n=5) were normalized to 100% and are shown without error bars. (B) Results are presented as pmol 5-FdUMP bound per mg total protein (×10⁻³) to reveal that transfection with control ODN 32 had no significant effect on TS protein levels. Error bars indicate errors calculated according to a Student t-test, and indicate error due to differences in experimental conditions in 5 measurements taken on different days, and differences due to transfection with different ODNs. The asterisks indicate significant differences (p<0.02) determined by using a paired Student t-test.

FIG. 13 shows that antisense TS ODN 83 sensitizes HeLa cells to the toxic effects of 5-FU, 5-FUdR, Tomudex, and MTX, but not cisplatin or chlorambucil. HeLa cells were transfected wraith ODN 83 (•) or control ODN 32 (∘) and treated with different concentrations of 5-FU (A), 5-FUdR (B), Tomudex (C), MTX (D), cisplatin (E) or chlorambucil (F) for 4 days, beginning 24 h after transfection. Data points are plotted as the mean±SE of 4 measurements. Where error bars are not apparent, they are obscured by the symbol. Asterisks (*) indicate significant differences <0.02, Student t-test).

FIG. 14A-F illustrates the effect of an antisense oligonucleotide targeting thymidylate synthase according to one embodiment of the invention on in vitro proliferation of various tumour cell lines;

FIG. 15 illustrates the effect of an antisense oligonucleotide targeting thymidylate synthase (TS) according to one embodiment of the invention on TS mRNA levels in tumour cells in vitro;

FIG. 16 illustrates the effectiveness of antisense oligonucleotide delivery in HeLa cells. (A) depicts micrographs of cells treated with control scrambled oligonucleotide or a TS antisense oligonucleotide, (B) shows the effect of varying incubation time with the oligonucleotide on delivery and (C) shows the effect of varying the concentration of oligonucleotide on delivery;

FIG. 17 compares uptake of antisense oligonucleotide in various tumour cell lines;

FIG. 18 illustrates the effect of treatment with thymidylate synthase (TS) antisense oligonucleotide in combination with TS-targeting chemotherapeutic agents on colon cancer (HT-29) cells;

FIG. 19A-E illustrates the effect of an antisense oligonucleotide targeting TS according to one embodiment of the invention on in Vitro proliferation of various tumour cell lines;

FIG. 20 illustrates the effect of an antisense oligonucleotide targeting TS according to one embodiment of the invention on TS mRNA levels in mesothelioma 211H, 2052, and H28 cells in vitro;

FIG. 21A-C illustrates the quantitation of RT-PCR products from mesothelioma 211H, 2052, and H28 cells treated with an antisense oligonucleotide of the invention targeting TS;

FIG. 22A-E illustrates the effect of an antisense oligonucleotide targeting TS according to one embodiment of the invention in combination with pemetrexed (Alimta®) on in vitro proliferation of various tumour cell lines;

FIG. 23A-D illustrates the effect of an antisense oligonucleotide targeting TS according to one embodiment of the invention in combination with 5-FUdR on in vitro proliferation of various tumour cell lines;

FIG. 24A-C illustrates the illustrates the effect of an antisense oligonucleotide targeting TS according to one embodiment of the invention in combination with gemcitabine on in vitro proliferation of various tumour cell lines; and

FIG. 25 provides the sequence of the human thymidylate synthase mRNA (SEQ ID NO:8).

FIG. 26 presents a graph indicating that ODN 83, at a dose which had no antitumor effect on its own, significantly enhanced the antitumor effect of pemetrexed (50 mg/kg). ODN 32 is the scrambled control. Mice were injected on both flanks with 7.5×10⁶ A549 human non-small cell lung carcinoma cells in matrigel. Tumours took approximately 3 weeks to reach 200 mm³. ODNs were administered IP at 25 mg/kg; pemetrexed at 50 mg/kg.

FIG. 26 presents graphs showing that ODN 83 alone, at 25 mg/kg, and pemetrexed alone, at 50 mg/kg, had no antitumor effect compared with control ODN 32. Doubling of tumor size, determined by interpolation of plotted data, is plotted as an event on a Kaplan-Meier curve. (A) Length of time for tumor to double in size in the presence of ODNs. (B) Length of time for tumor to double in size in presence or absence of pemetrexed.

FIG. 27 presents graphs showing that the combination of ODN 83 and pemetrexed significantly inhibited tumor growth to 200% of starting volume compared to ODN 83 alone or pemetrexed alone. (A) Synergistic inhibition by ODN 83/pemetrexed combination compared to ODN 83 alone. (B) Synergistic inhibition by ODN 83/pemetrexed combination compared to pemetrexed alone. (C) Synergistic inhibition by ODN 83/pemetrexed combination compared to ODN 32/pemetrexed.

FIG. 28 presents a bar graph showing that the combination of ODN 83 and pemetrexed synergistically slowed tumor growth rates. 25 mg/kg ODN 083 and 50 mg/kg pemetrexed had no effect on their own, compared with control ODN 32.

### DETAILED DESCRIPTION OF THE INVENTION

In a first aspect of the invention, there is provided an antisense oligodeoxynucleotide which hybridises to a target nucleic acid sequence in thymidylate synthase and which selectively inhibits thymidylate synthase production in mammalian cells. Preferably the oligonucleotide is targeted to sequences at or near the translational stop site at the 3′ end of the TS gene, which sequences lie in the region between bases 800 and 1600, using the sequence numbering described for human thymidylate synthase mRNA by Takeishi et al. (Takeishi et al., 1985, Nucleic Acids Res. 13: 2035-2043). More preferably the sequences lie in the region between bases 1000 and 1530. Most preferably the sequences lie in the region between bases 1030 and 1460.

In a further aspect of the present invention, there is provided a combination product comprising an antisense oligonucleotide targeted to thymidylate synthase in combination with an anticancer agent. The antisense oligonucleotide and the anticancer agent may be administered separately, sequentially, simultaneously or in a mixture.

In one embodiment of the invention, there is provided a combination product comprising an antisense oligonucleotide targeted to thymidylate synthase (TS) in combination with a chemotherapeutic agent that also targets TS. In another embodiment, the invention provides for a combination product comprising an antisense oligonucleotide having a sequence that comprises at least 10 consecutive nucleotides of SEQ ID NO:1 in combination with a chemotherapeutic agent that targets TS. In a further embodiment, the invention provides a combination product comprising an antisense oligonucleotide targeted to TS in combination with a chemotherapeutic agent that also targets TS selected from the group of 5-fluorouracil, capecitabine, raltitrexed and pemetrexed.

In a further aspect of the present invention there is provided a pharmaceutical composition comprising a combination product as defined hereinbefore and a pharmaceutically acceptable diluent or carrier.

In a further aspect of the invention there is provided a method for the treatment of cancer (or a method for providing an antiproliferative effect) which comprises administering to a warm-blooded animal an effective amount of a combination product as defined above. The invention also provides the use of such a combination product in the production of a new medicam ent for the treatment of cancer (or for the treatment of proliferative disease).

### DEFINITIONS

Unless defined otherwise, all technical and scientific terms used herein have the same meaning as commonly understood by one of ordinary skill in the art to which this invention pertains.

The terms “antisense oligonucleotide” and, “antisense oligodeoxynucleotide” (ODN) as used herein refer to a nucleotide sequence that is complementary to a mRNA for a target gene. In the context of the present invention, the target gene is the gene encoding a mammalian thymidylate synthase protein.

The term “selectively hybridise” as used herein refers to the ability of a nucleic acid to bind detectably and specifically to a second nucleic acid. Oligonucleotides selectively hybridise to target nucleic acid strands under hybridisation and wash conditions that minimise appreciable amounts of detectable binding to non-specific nucleic acids. High stringency conditions can be used to achieve specific hybridization conditions as known in the art. Typically, hybridization and washing are performed at high stringency according to conventional hybridization procedures and employing one or more washing step in a solution comprising 1-3×SSC, 0.1-1% SDS at 50-70° C. for 5-30 minutes.

The term “corresponds to” as used herein with reference to nucleic acid sequences means a polynucleotide sequence that is identical to all or a portion of a reference polynucleotide sequence. In contradistinction, the term “complementary to” is used herein to mean that the polynucleotide sequence is identical to all or a portion of the complement of a reference polynucleotide sequence. For illustration, the nucleotide sequence “TATAC” corresponds to a reference sequence “TATAC” and is complementary to a reference sequence “GTATA”.

The following terms are used herein to describe the sequence relationships between two or more polynucleotides: “reference sequence,” “window of comparison,” “sequence identity,” “percent sequence identity,” and “substantial identity.” A “reference sequence” is a defined sequence used as a basis for a sequence comparison; a reference sequence may be a subset of a larger sequence, for example, as a segment of a full-length cDNA, gene sequence, or may comprise a complete cDNA, gene sequence. Generally, a reference polynucleotide sequence is at least 20 nucleotides in length, and often at least 50 nucleotides in length.

A “window of comparison”, as used herein, refers to a conceptual segment of the reference sequence of at least 15 contiguous nucleotide positions over which a candidate sequence may be compared to the reference sequence and wherein the portion of the candidate sequence in the window of comparison may comprise additions or deletions (i.e. gaps) of 20 percent or less as compared to the reference sequence (which does not comprise additions or deletions) for optimal alignment of the two sequences. The present invention contemplates various lengths for the window of comparison, up to and including the full length of either the reference or candidate sequence. Optimal alignment of sequences for aligning a comparison window may be conducted using the local homology algorithm of Smith and Waterman (Adv. Appl. Math. (1981) 2:482), the homology alignment algorithm of Needleman and Wunsch (J. Mol. Biol. (1970) 48:443), the search for similarity method of Pearson and Lipman (Proc. Ncitl. Acad. Sci. (U.S.A.) (1988) 85:2444), using computerised implementations of these algorithms (such as GAP, BESTFIT, FASTA, and TFASTA in the Wisconsin Genetics Software Package Release 7.0, Genetics Computer Group, 573 Science Dr., Madison, Wis.), using publicly available computer software such as ALIGN or Megalign (DNASTAR), or by inspection. The best alignment (i.e. resulting in the highest percentage of identity over the comparison window) is then selected.

The term “sequence identity” means that two polynucleotide sequences are identical (i.e. on a nucleotide-by-nucleotide basis) over the window of comparison.

The term “percent (%) sequence identity,” as used herein with respect to a reference sequence is defined as the percentage of nucleotide residues in a candidate sequence that are identical with the residues in the reference sequence over the window of comparison after optimal alignment of the sequences and introducing gaps, if necessary, to achieve the maximum percent sequence identity, without considering any conservative substitutions as part of the sequence identity.

The term “substantial identity” as used herein denotes a characteristic of a polynucleotide sequence, wherein the polynucleotide comprises a sequence that has at least 50% sequence identity as compared to a reference sequence over the window of comparison. Polynucleotide sequences at least 60% sequence identity, at least 70% sequence identity, at least 80% sequence identity, and at least 90% sequence identity as compared to a reference sequence over the window of comparison are also considered to have substantial identity with the reference sequence.

The terms “therapy,” and “treatment,” as used interchangeably herein, refer to an intervention performed with the intention of improving a recipient's status. The improvement can be subjective or objective and is related to the amelioration of the symptoms associated with, preventing the development of, or altering the pathology of a disease, disorder or condition being treated. Thus, the terms therapy and treatment are used in the broadest sense, and include the prevention (prophylaxis), moderation, reduction, and curing of a disease, disorder or condition at various stages. Prevention of deterioration of a recipient's status is also encompassed by the term. Those in need of therapy/treatment include those already having the disease, disorder or condition as well as those prone to, or at risk of developing, the disease, disorder or condition and those in whom the disease, disorder or condition is to be prevented.

The term “ameliorate” or “amelioration” includes the arrest, prevention, decrease, or improvement in one or more the symptoms, signs, and features of the disease being treated, both temporary and long-term.

The term “subject” or “patient” as used herein refers to an animal in need of treatment.

The term “animal,” as used herein, refers to both human and non-human animals, including, but not limited to, mammals, birds and fish.

Administration of an antisense oligonucleotide “in combination with” one or more anticancer agents, is intended to include simultaneous (concurrent) administration and consecutive administration. Consecutive administration is intended to encompass administration of the therapeutic agent(s) and the compound(s) of the invention to the subject in various orders and via various routes.

The term “combination product” as used in the context of the present invention refers to a product that includes an antisense oligonucleotide and an anticancer agent, such as a chemotherapeutic agent. The combination product can be provided in various forms, for example, as a combination of two separate pharmaceutical compositions, each comprising one component of the combination product, which may optionally be provided in dosage units; or, when the components are compatible, as a single formulation comprising both components, which formulation may optionally be provided in dosage units.

As used herein, the term “about” refers to approximately a +/−10% variation from a given value. It is to be understood that such a variation is always included in any given value provided herein, whether or not it is specifically referred to.

### Antisense Oligonucleotides

### Selection and Characteristics

An antisense oligodeoxynucleotide is an oligonucleotide which is designed to hybridise to a specific region of a target nucleic acid sequence. The target nucleic acid is the thymidylate synthase (TS) gene or mRNA transcribed from the TS gene. Preferably the target nucleic acid is the mRNA encoding thymidylate synthase. Various antisense oligonucleotides against TS and combinations of these antisense with chemotherapeutics are described in U.S. Pat. No. 6,087,489, and U.S. patent application Ser. Nos. 09/509,418 and 10/597,409, which are all expressly and specifically incorporated by reference in their entirety.

The sequences of various mammalian TS genes and mRNAs are known in the art and can be readily obtained from Genbank (maintained by the National Center for Biotechnology Information). In one embodiment of the invention, the antisense oligonucleotides are targeted to the human TS gene. In another embodiment, the antisense oligonucleotides comprise a sequence complementary to a portion of the human TS miRNA. The sequence for human TS miRNA can be accessed from GenBank under Accession No. X02308 and is provided herein as FIG. 25 (SEQ ID NO:8).

In targeting the antisense to the TS gene or mRNA a determination is made of a site or sites within this gene or its mRNA for the antisense interaction to occur such that the desired effect, i.e. modulation of expression of the protein encoded by the gene, will result. Once the target site or sites have been identified, oligonucleotides are chosen that are sufficiently complementary (i.e. hybridise with sufficient strength and specificity) to the target to give the desired result. Hybridisation of an antisense oligonucleotide to its target nucleic acid sequence is mediated by the formation of hydrogen bonds between complementary bases on each nucleic acid strand. Hybridisation may occur between nucleic acid strands which have varying degrees of complementarity, depending on the hybridisation conditions employed. The term “specifically hybridisable” is used to describe an oligonucleotide which has a sufficient degree of complementarity to ensure stable, specific binding to its target sequence, whilst avoiding non-specific binding to non-target sequences.

Hybridisation of the antisense oligonucleotide to thymidylate synthase mRNA may affect one or more aspects of mRNA function, for example mRNA translocation, mRNA splicing, mRNA translation, or the feedback inhibition mechanism regulated by the binding of thymidylate synthase protein to binding sites within the thymidylate synthase mRNA molecule.

Antisense oligonucleotides may be designed to hybridise to various regions within the thymidylate synthase mRNA molecule, including the coding region, the 5′ untranslated region (5′-UTR), the 3′ untranslated region (3′-UTR), the 5′ cap region, introns and intron/exon splice junctions. In one embodiment, the antisense oligonucleotide is targeted to the 5′-UTR, the translation initiation or start codon region, the open reading frame (ORF), the translation termination or stop codon region or the 3′-UTR. In accordance with one embodiment of the present invention, the antisense oligonucleotide is targeted to part of the 3′-UTR of the TS gene.

The antisense oligonucleotides in accordance with the present invention are selected from a sequence complementary to the TS gene or mRNA such that the sequence exhibits the least likelihood of forming duplexes, hair-pins, or of containing homooligomer/sequence repeats. The oligonucleotide may further contain a GC clamp. One skilled in the art will appreciate that these properties can be determined qualitatively using various computer modelling programs, for example, the program OLIGO® Primer Analysis Software, Version 5.0 (distributed by National Biosciences, Inc., Plymouth, Minn.).

It is understood in the art that an antisense oligonucleotide need not have 100% identity with the complement of its target sequence. The antisense oligonucleotides in accordance with the present invention have a sequence that is at least about 75% identical to the complement of target sequence. In one embodiment of the present invention, the antisense oligonucleotides have a sequence that is at least about 90% identical to the complement of the target sequence. In a related embodiment, they have a sequence that is at least about 95% identical to the complement of target sequence, allowing for gaps or mismatches of several bases. Identity can be determined, for example, by using the BLASTN program of the University of Wisconsin Computer Group (GCG) software or provided on the NCBI website.

In accordance with the present invention, antisense oligonucleotides comprise at least 7 consecutive nucleotides complementary to a part of a mammalian TS gene and are typically between 7 and 100 nucleotides in length. In one embodiment, the antisense oligonucleotides are between about 7 to about 50 nucleotides in length. In another embodiment, the antisense oligonucleotides are between about 7 to about 35′ nucleotides in length. In a further embodiment, the antisense oligonucleotides are between about 15 to about 25 nucleotides in length.

In another embodiment, the antisense oligonucleotides are from about 8 to about 50 nucleotides in length, more preferably from about 12 to about 40 nucleotides in length and most preferably from about 16 to about 30 nucleotides in length. In a further embodiment, the antisense oligonucleotides comprise at least 10 consecutive nucleotides complementary to a part of a mammalian TS gene and are typically between 10 and 100 nucleotides in length. In another embodiment, the antisense oligonucleotides are between about 10 to about 50 nucleotides in length.

Exemplary, non-limiting examples of sequences for antisense oligonucleotides against a human TS gene are provided in Tables 1 and 2.

TABLE 1
Exemplary Oligonucleotides (ODNs) Targeted to Human TS

| | Target Sequence | ODN Sequence (5′→3′) |
|---|---|---|
| ODN | (SEQ ID NO) | (SEQ ID NO) |
| OLIGO 83 | TTAAGGATGTTGCCACTGGC (9) | GCCAGTGGCAACATCCTTAA (1) |
| OLIGO 86 | AATGGCTGTTTAGGGTGCTT (10) | AAGCACCCTAAACAGCCATT (2) |
| OLIGO 90 | TGTGGCCGGCTCGGAGCTGC (11) | GCAGCTCCGAGCCGGCCACA (3) |
| OLIGO 91 | GCGCCATGCCTGTGGCCGGC (12) | GCCGGCCACAGGCATGGCGC (4) |
| OLIGO 92 | GCGCCATGCCTGTGGCCGGC (13) | GCCGGCCACAGGCATGGCGC (4) |
| OLIGO 93 | CCCGCCCGCCGCGCCATGCC (14) | GGCATGGCGCGGCGGGCGGG (5) |
| OLIGO 81 | CAAATCTGAGGGAGCTGAG (15) | CTCAGCTCCCTCAGATTTG (6) |
| ODN 32 | — | ATGCGCCAACGGTTCCTAAA (7) |
| PAS/TSS | UGUGGCCGGCUCGGAGCUGCCGCGCCGGCC (16) | |
| PAS/EXON 1, 2 | GCUACAGCCUGAGAGAUGAAUUCCCUCUGC (17) | |

TABLE 2
Exemplary Antisense ODNs against Human TS

| | | |
|---|---|---|
| | Complementary | |
| Sequence (5′→3′) | Region in TS mRNA | SEQ ID NO |

| | | |
|---|---|---|
| GCCAGTGGCAACATCCTTAA | 1184-1203 | 1 |
| TTGGATGCGGATTGTACCCT | 1002-1021 | 18 |
| ACTCAGCTCCCTCAGATTTG | 1436-1455 | 19 |
| CCAGCCCAACCCCTAAAGAC | 1081-1100 | 20 |
| GGCATCCCAGATTTTCACTC | 419-438 | 21 |
| AGCATTTGTGGATCCCTTGA | 380-399 | 22 |

In one embodiment of the present invention, the antisense oligonucleotides comprise at least 7 consecutive nucleotides of the sequence as set forth in any one of SEQ ID NOs: 1, 18, 19, 20, 21 or 22. In another embodiment, the antisense oligonucleotides comprise at least 10 consecutive nucleotides of the sequence as set forth in any one of SEQ ID NOs: 1, 18, 19, 20, 21 or 22.

It will be appreciated that the invention is not restricted merely to those specific antisense oligonucleotides which are disclosed in Tables 1 and 2 above but encompasses oligonucleotides of from about 7 to about 100 nucleotides in length, for example from about 8 to about 50 nucleotides in length, which selectively inhibit or selectively enhance thymidylate synthase production and which are selected from those regions of the TS gene which are described hereinbefore.

The term “antisense oligonucleotides” as used herein includes other oligomeric antisense compounds, including oligonucleotide mimetics, modified oligonucleotides, and chimeric antisense compounds. Chimeric antisense compounds are antisense compounds that contain two or more chemically distinct regions, each made up of at least one monomer unit.

Thus, in the context of this invention, the term “oligonucleotide” refers to an oligomer or polymer of ribonucleic acid (RNA), deoxyribonucleic acid (DNA), or RNA or DNA mimetics. This term, therefore, includes oligonucleotides composed of naturally-occurring nucleobases, sugars and covalent internucleoside (backbone) linkages as well as oligonucleotides having non-naturally-occurring portions, which function similarly. Such modified or substituted oligonucleotides are often preferred over native forms because of desirable properties such as, for example, enhanced cellular uptake, enhanced affinity for nucleic acid target and increased stability in the presence of nucleases.

As is known in the art, a nucleoside is a base-sugar combination and a nucleotide is a nucleoside that further includes a phosphate group covalently linked to the sugar portion of the nucleoside. In forming oligonucleotides, the phosphate groups covalently link adjacent nucleosides to one another to form a linear polymeric compound, with the normal linkage or backbone of RNA and DNA being a 3′ to 5′ phosphodiester linkage. Specific examples of antisense compounds useful in this invention include oligonucleotides containing modified backbones or non-natural internucleoside linkages. As defined in this specification, oligonucleotides having modified backbones include both those that retain a phosphorus atom in the backbone and those that lack a phosphorus atom in the backbone. For the purposes of the present invention, and as sometimes referenced in the art, modified oligonucleotides that do not have a phosphorus atom in their internucleoside backbone can also be considered to be oligonucleotides.

Exemplary modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

In one embodiment of the present invention, the antisense oligonucleotide comprises at least one phosphorothioate linkage. In another embodiment, the antisense oligonucleotide comprises phosphorothioate internucleotide linkages that link the four, five or six 3′-terminal nucleotides of the oligonucleotide. In a further embodiment, the antisense oligonucleotide comprises phosphorothioate internucleotide linkages that link all the nucleotides of the oligonucleotide.

Exemplary modified oligonucleotide backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include morpholino linkages (formed in part from the sugar portion of a nucleoside); siloxane backbones; sulfide, sulfoxide and sulphone backbones; formacetyl and thioformacetyl backbones; methylene formacetyl and thioformacetyl backbones; alkene containing backbones; sulphamate backbones; methyleneimino and methylenehydrazino backbones; sulphonate and sulfonamide backbones; amide backbones; and others having mixed N, O, S and CH₂ component parts.

The present invention also contemplates oligonucleotide mimetics in which both the sugar and the internucleoside linkage of the nucleotide units are replaced with novel groups. The base units are maintained for hybridisation with an appropriate nucleic acid target compound. An example of such an oligonucleotide mimetic, which has been shown to have excellent hybridisation properties, is a peptide nucleic acid (PNA) [Nielsen et al., Science, 254:1497-1500 (1991)]. In PNA compounds, the sugar-backbone of an oligonucleotide is replaced with an amide containing backbone, in particular an aminoethylglycine backbone. The nucleobases are retained and are bound directly or indirectly to aza-nitrogen atoms of the amide portion of the backbone.

The present invention also contemplates oligonucleotides comprising “locked nucleic acids” (LNAs), which are novel conformationally restricted oligonucleotide analogues containing a methylene bridge that connects the 2′-O of ribose with the 4′-C (see, Singh et al., Chem. Commun., 1998, 4:455-456). LNA oligonucleotides contain one or more nucleotide building blocks in which an extra methylene bridge, as noted above, that fixes the ribose moiety either in the C3′-endo (β-D-LNA) or C2′-endo (α-L-LNA) conformation.

LNA and LNA analogues display very high duplex thermal stabilities with complementary DNA and RNA, stability towards 3′-exonuclease degradation, and good solubility properties. Synthesis of the LNA analogues of adenine, cytosine, guanine, 5-methylcytosine, thymine and uracil, their oligomerization, and nucleic acid recognition properties have been described (see Koshkin et al., Tetrahedron, 1998, 54:3607-3630). Studies of mis-matched sequences show that LNA obey the Watson-Crick base pairing rules with generally improved selectivity compared to the corresponding unmodified reference strands.

Antisense oligonucleotides containing LNAs have been described (Wahlestedt et al., Proc. Natl. Acad. Sci. U.S.A., 2000, 97:5633-5638), which were efficacious and nontoxic. In addition, the LNA/DNA copolymers were not degraded readily in blood serum and cell extracts. LNAs form duplexes with complementary DNA or RNA or with complementary LNA, with high thermal affinities. The universality of LNA-mediated hybridization has been emphasized by the formation of exceedingly stable LNA:LNA duplexes (Koshkin et al., J. Am. Chem. Soc., 1998, 120:13252-13253). LNA:LNA hybridization was shown to be the most thermally stable nucleic acid type duplex system, and the RNA-mimicking character of LNA was established at the duplex level. Introduction of three LNA monomers (T or A) resulted in significantly increased melting points toward DNA complements.

Synthesis of 2′-amino-LNA (Singh et al., J. Org. Chem., 1998, 63, 10035-10039) and 2′-methylamino-LNA has been described and thermal stability of their duplexes with complementary RNA and DNA strands reported. Preparation of phosphorothioate-LNA and 2′-thio-LNA have also been described (Kumar et al., Bioorg. Med. Chem. Lett., 1998, 8:2219-2222).

The antisense oligonucleotides comprising LNAs can be designed as “gapmers” in which the oligonucleotide comprises a stretch of LNAs at the 5′ end, followed by a “gap” of DNA nucleotides, then a second stretch of LNAs at the 3′ end.

In one embodiment, the antisense oligonucleotides of the invention comprise LNAs. In another embodiment, the antisense oligonucleotides of the invention comprise β-D-LNAs. In a further embodiment, antisense oligonucleotides of the invention are LNA gapmers, as described above.

Modified oligonucleotides may also contain one or more substituted sugar moieties. For example, oligonucleotides may comprise sugars with one of the following substituents at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Examples of such groups are: O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Alternatively, the oligonucleotides may comprise one of the following substituents at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkyl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Specific examples include 2′-methoxyethoxy (2′-O—CH₂, CH₂, OCH₃, also known as 2′-O-(2-methoxyethyl) or 2′-MOE) [Martin et al., Helv. Chim. Acta, 78:486-504 (1995)], 2′-dimethylaminooxyethoxy (O(CH₂)₂ ON(CH₃)₂ group, also known as 2′-DMAOE), 2′-methoxy (2′-O—CH₃), 2′-aminopropoxy (2′-OCH₂CH₂CH₂NH₂) and 2′-fluoro (2′-F).

In one embodiment of the present invention, the antisense oligonucleotide comprises at least one nucleotide comprising a substituted sugar moiety. In another embodiment, the antisense oligonucleotide comprises at least one 2′-O-(2-methoxyethyl) or 2′-MOE modified nucleotide.

Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include modifications or substitutions to the nucleobase. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl, and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808; The Concise Encyclopedia Of Polymer Science And Engineering, (1990) pp 858-859, Kroschwitz, J. I., ed. John Wiley & Sons; Englisch et al., Angewandte Chemie. Int. Ed., 30:613 (1991); and Sanghvi, Y. S., (1993) Antisense Research and Applications, pp 289-302, Crooke, S. T. and Lebleu, B., ed., CRC Press. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi, Y. S., (1993) Antisense Research and Applications, pp 276-278, Crooke, S. T. and Lebleu, B., ed., CRC Press, Boca Raton].

Another oligonucleotide modification included in the present invention is the chemically linkage to the oligonucleotide of one or more moieties or conjugates which enhance the activity, cellular distribution or cellular uptake of the oligonucleotide. Such moieties include, but are not limited to, lipid moieties such as a cholesterol moiety [Letsinger et al., Proc. Natl. Acad. Sci. USA, 86:6553-6556 (1989)], cholic acid [Manoharan et al., Bioorg. Med. Chem. Let., 4:1053-1060 (1994)], a thioether, e.g. hexyl-S-tritylthiol [Manoharan et al., Ann. N.Y. Acad. Sci., 660:306-309 (1992); Manoharan et al., Bioorg. Med. Chem. Lett., 3:2765-2770 (1993)], a thiocholesterol [Oberhauser et al., Nucl. Acids Res., 20:533-538 (1992)], an aliphatic chain, e.g. dodecandiol or undecyl residues [Saison-Behmoaras et al., EMBO J., 10:1111-1118 (1991); Kabanov et al., FEBS Lett., 259:327-330 (1990); Svinarchuk et al., Biochimie, 75:49-54 (1993)], a phospholipid, e.g. di-hexadecyl-rac-glycerol or triethylammonium 1,2-di-O-hexadecyl-rac-glycero-3-H-phosphonate [Manoharan et al., Tetrahedron Lett., 36:3651-3654 (1995); Shea et al., Nucl. Acids Res., 18:3777-3783 (1990)], a polyamine or a polyethylene glycol chain [Manoharan et al., Nucleosides & Nucleotides, 14:969-973 (1995)], or adamantane acetic acid [Manoharan et al., Tetrahedron Lett., 36:3651-3654 (1995)], a palmityl moiety [Mishra et al., Biochim. Biophys. Acta, 1264:229-237 (1995)], or an octadecylamine or hexylamino-carbonyl-oxycholesterol moiety [Crooke et al., J. Pharmacol. Exp. Ther., 277:923-937 (1996)].

One skilled in the art will recognise that it is not necessary for all positions in a given oligonucleotide to be uniformly modified. The present invention, therefore, contemplates the incorporation of more than one of the aforementioned modifications into a single oligonucleotide or even at a single nucleoside within the oligonucleotide. The present invention thus further includes antisense compounds that are chimeric compounds. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridising to the same target region. Cleavage of the RNA target can be routinely detected by gel electrophoresis and, if necessary, associated nucleic acid hybridisation techniques known in the art.

In one embodiment of the present invention, the antisense oligonucleotides comprise a phosphorothioate backbone in combination with at least one 2′-MOE modified sugar. In another embodiment, the antisense oligonucleotides comprise a phosphorothioate backbone in combination with one or more 2′-MOE modified sugars at the 3′ and 5′ ends of the oligonucleotide. In a further embodiment, the antisense oligonucleotides are “methoxy-ethoxy winged” oligonucleotides with one or more 2′-MOE modified sugars at the 3′ and 5′ ends of the oligonucleotide.

In the context of the present invention, an antisense oligonucleotide is “nuclease resistant” when it has either been modified such that it is less susceptible to degradation by DNA and/or RNA nucleases than when in its non-modified form or alternatively has been placed in a delivery vehicle which in itself protects the oligonucleotide from DNA or RNA nucleases. Nuclease resistant oligonucleotides include, for example, methyl phosphonates, phosphorothioates, phosphorodithioates, phosphotriesters, and morpholino oligomers. Suitable delivery vehicles for conferring nuclease resistance include, for example, liposomes. In one embodiment of the present invention, the antisense oligonucleotides are nuclease resistant.

The present invention further contemplates antisense oligonucleotides that contain groups for improving the pharmacokinetic properties of the oligonucleotide, or groups for improving the pharmacodynamic properties of the oligonucleotide.

Short Interfering RNA (siRNA) Molecules

The present invention further contemplates that the antisense oligonucleotides may be in the form of siRNA molecules. RNA interference mediated by double-stranded siRNA molecules, which are generated in nature when long double-stranded RNA molecules are cleaved by the action of an endogenous ribonuclease, is known in the art to play an important role in post-transcriptional gene silencing [Zamore, Nature Struc. Biol., 8:746-750 (2001)]. Transfection of mammalian cells with synthetic siRNA molecules having a sequence identical to a target gene has been demonstrated to result in a reduction in the mRNA levels of the target gene [see, for example, Elbashir, et al., Nature, 411:494-498 (2001)]. siRNA molecules are typically 21-22 base pairs in length.

The specificity of siRNA molecules is determined by the binding of the antisense strand of the molecule to its target mRNA. Thus, the antisense oligonucleotides of the present invention can be provided as part of an siRNA molecule which is targeted to a TS gene. As is known in the art, effective siRNA molecules should be less than 30 to 35 base pairs in length to prevent them triggering non-specific RNA interference pathways in the cell via the interferon response. Thus, in one embodiment of the present invention, the siRNA molecules are between about 15 and about 25 base pairs in length. In a related embodiment, they are between 19 and 22 base pairs in length.

The double-stranded siRNA molecules can further comprise poly-T or poly-U overhangs at each end to minimise RNase-mediated degradation of the molecules. In another embodiment of the present invention, the siRNA molecules comprise overhangs at the 3′ and 5′ ends which consist of two thymidine or two uridine residues. Design and construction of siRNA molecules is known in the art [see, for example, Elbashir, et al., Nature, 411:494-498 (2001); Bitko and Barik, BMC Microbiol., 1:34 (2001)]. In addition, kits that provide a rapid and efficient means of constructing siRNA molecules by in vitro transcription are also commercially available (Ambion, Austin, Tex.; New England Biolabs, Beverly, Mass.).

Single-stranded siRNA and short-hairpin siRNA (shRNA) molecules are also known in the art. The present invention contemplates that the antisense oligonucleotides against TS can be provided as single-stranded siRNA molecules and as shRNA molecules.

siRNA molecules can comprise naturally-occurring nucleobases, sugars and covalent internucleoside (backbone) linkages as well as oligonucleotides having non-naturally-occurring portions, which function similarly, as described above.

### Preparation of the Antisense Oligonucleotides

The antisense oligonucleotides of the present invention can be prepared by conventional techniques well-known to those skilled in the art. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1^{st} Edition).

For example, the oligonucleotides can be prepared using solid-phase synthesis using commercially available equipment, such as the equipment available from Applied Biosystems Canada Inc., Mississauga, Canada. As is well-known in the art, modified oligonucleotides, such as phosphorothioates and alkylated derivatives, can also be readily prepared by similar methods.

Alternatively, the antisense oligonucleotides of the present invention can be prepared by enzymatic digestion of the naturally occurring thymidylate synthase gene by methods known in the art.

Antisense oligonucleotides can also be prepared through the use of recombinant methods in which expression vectors comprising nucleic acid sequences that encode the antisense oligonucleotides are expressed in a suitable host cell. Such expression vectors can be readily constructed using procedures known in the art. Examples of suitable vectors include, but are not limited to, plasmids, phagemids, cosmids, bacteriophages, baculoviruses and retroviruses, and DNA viruses. One skilled in the art will understand that selection of the appropriate host cell for expression of the antisense oligonucleotide will be dependent upon the vector chosen. Examples of host cells include, but are not limited to, bacterial, yeast, insect, plant and mammalian cells.

One skilled in the art will also understand that the expression vector may further include regulatory elements, such as transcriptional elements, required for efficient transcription of the antisense oligonucleotide sequences. Examples of regulatory elements that can be incorporated into the vector include, but are not limited to, promoters, enhancers, terminators, and polyadenylation signals. One skilled in the art will appreciate that selection of suitable regulatory elements is dependent on the host cell chosen for expression of the antisense oligonucleotide and that such regulatory elements may be derived from a variety of sources, including, bacterial, fungal, viral, mammalian or insect genes.

In accordance with the present invention, the expression vectors can be introduced into a suitable host cell or tissue by one of a variety of methods known in the art. Such methods can be found generally described in Sambrook et al., 1992; Ausubel et al., 1989; Chang et al., 1995; Vega et al. 1995; and Vectors: A Survey of Molecular Cloning Vectors and Their Uses (1988) and include, for example, stable or transient transfection, lipofection, electroporation, and infection with recombinant viral vectors.

### Efficacy of the Antisense Oligonucleotides

The effects of antisense oligonucleotides on thymidylate synthase expression can be measured using procedures which are well known to persons skilled in the art. In the present application, effects on mRNA levels have been measured by Northern blot analysis and nuclear run-on transcription assay, and effects on the growth of human tumour cells have been measured by counting cell numbers using a Coulter counter. Other methods are known in the art.

Antisense oligonucleotides to thymidylate synthase may inhibit, stimulate or have no effect on thymidylate synthase expression. Of these, preferred antisense oligonucleotides are those which either inhibit or stimulate thymidylate synthase expression, and particularly preferred antisense oligonucleotides are those which inhibit thymidylate synthase expression.

By inhibition of thymidylate synthase expression we mean inhibition of at least 10% relative to the untreated control, measured at day 4 using the assay described in Example 1.2. Preferably inhibition of thymidylate synthase expression is at least 20% and most preferably inhibition is at least 40%.

### 1. In Vitro Testing

Initial determinations of the efficacy of the antisense oligonucleotides of the invention alone, or in combination with one or more chemotherapeutic, can be made using in vitro techniques, if required.

For example, the ability of the antisense oligonucleotides to inhibit proliferation of cancer cells can be assessed by culturing cells of a cancer cell line of interest in a suitable medium. After an appropriate incubation time, the cells can be transfected with the antisense oligonucleotide, for example in the presence of a commercial lipid carrier such as lipofectamine, and incubated for a further period of time. Cells are then counted and compared to an appropriate control. Suitable controls include, for example, cells treated with a control oligonucleotide (such as a scrambled form of the test oligonucleotide), cells treated with a standard chemotherapeutic and/or untreated cells.

Alternatively, the antisense oligonucleotides can be tested in vitro by determining their ability to inhibit anchorage-independent growth of tumour cells. Anchorage-independent growth is known in the art to be a good indicator of tumorigenicity. In general, anchorage-independent growth is assessed by plating cells from a selected cancer cell-line onto soft agar and determining the number of colonies formed after an appropriate incubation period. Growth of cells treated with the antisense oligonucleotide can then be compared with that of control cells (as described above).

Similar methods can be employed to test the efficacy of the antisense oligonucleotides in combination with chemotherapeutic(s). Suitable controls in this case would include cells treated with the antisense oligonucleotide alone and cells treated with the chemotherapeutic(s) alone.

In one embodiment of the present invention, in vitro testing of the antisense oligonucleotides is conducted in a human cancer cell-line. Examples of suitable cancer cell-lines for in vitro testing include, but are not limited to, mesothelial cell lines MSTO-211H, NCI-H2052 and NCI-H28, ovarian cancer cell-lines OV90 and SK-OV-3, breast cancer cell-lines MCF-7 and MDA-MB-231, colon cancer cell-lines CaCo, HCT116 and HT29, cervical cancer cell-line HeLa, non-small cell lung carcinoma cell-lines A549 and H1299, pancreatic cancer cell-lines MIA-PaCa-2 and AsPC-1, prostatic cancer-cell line PC-3, bladder cancer cell-line T24, liver cancer cell-lineHepG2, brain cancer cell-line U-87 MG, melanoma cell-line A2058, lung cancer cell-line NCI-H460. Other examples of suitable cell-lines are known in the art.

If necessary, the toxicity of the antisense oligonucleotides can also be initially assessed in vitro using standard techniques. For example, human primary fibroblasts can be transfected in vitro with the oligonucleotide and then tested at different time points following treatment for their viability using a standard viability assay, such as the trypan-blue exclusion assay. Cells can also be assayed for their ability to synthesize DNA, for example, using a thymidine incorporation assay, and for changes in cell cycle dynamics, for example, using a standard cell sorting assay in conjunction with a fluorocytometer cell sorter (FACS).

### 2. In Vivo Testing

The ability of the antisense oligonucleotides, alone or in combination with one or more anticancer agents, to inhibit tumour growth or proliferation in vivo can be determined in an appropriate animal model using standard techniques known in the art (see, for example, Enna, et al., Current Protocols in Pharmacology, J. Wiley & Sons, Inc., New York, N.Y.).

In general, current animal models for screening anti-tumour compounds are xenograft models, in which a human tumour has been implanted into an animal. Examples of xenograft models of human cancer include, but are not limited to, human solid tumor xenografts in mice, implanted by sub-cutaneous injection and used in tumour growth assays; human solid tumour orthotopic xenografts, implanted directly into the relevant tissue and used in tumour growth assays; human solid tumour isografts in mice, implanted by fat pad injection and used in tumour growth assays; experimental models of lymphoma and leukaemia in mice, used in survival assays, and experimental models of lung metastasis in mice.

For example, the antisense oligonucleotides can be tested in vivo on solid tumours using mice that are subcutaneously grafted bilaterally with 30 to 60 mg of a tumour fragment, or implanted with an appropriate number of cancer cells, on day 0. The animals bearing tumours are mixed before being subjected to the various treatments and controls. In the case of treatment of advanced tumours, tumours are allowed to develop to the desired size, animals having insufficiently developed tumours being eliminated. The selected animals are distributed at random to undergo the treatments and controls. Animals not bearing tumors may also be subjected to the same treatments as the tumour-bearing animals in order to be able to dissociate the toxic effect from the specific effect on the tumour. Chemotherapy generally begins from 3 to 22 clays after grafting, depending on the type of tumour, and the animals are observed every day. The antisense oligonucleotide of the present invention can be administered to the animals, for example, by i.p. injection or bolus infusion. The different animal groups are weighed about 3 or 4 times a week until the maximum weight loss is attained, after which the groups are weighed at least once a week until the end of the trial.

The tumours are measured after a pre-determined time period, or they can be monitored continuously by measuring about 2 or 3 times a week until the tumour reaches a pre-determined size and/or weight, or until the animal dies if this occurs before the tumour reaches the pre-determined size/weight. The animals are then sacrificed and the tissue histology, size and/or proliferation of the tumour assessed.

Orthotopic xenograft models are an alternative to subcutaneous models and may more accurately reflect the cancer development process. In this model, tumour cells are implanted at the site of the organ of origin and develop internally. Daily evaluation of the size of the tumours is thus more difficult than in a subcutaneous model. A recently developed technique using green fluorescent protein (GFP) expressing tumours in non-invasive whole-body imaging can help to address this issue (Yang and al, Proc. Nat. Aca. Sci. (2000), pp 1206-1211). This technique utilises human or murine tumours that stably express very high levels of the Aqueora vitoria green fluorescent protein. The GFP expressing tumours can be visualised by means of externally placed video detectors, allowing for monitoring of details of tumour growth, angiogenesis and metastatic spread. Angiogenesis can be measured over time by monitoring the blood vessel density within the tumour(s). The use of this model thus allows for simultaneous monitoring od several features associated with tumour progression and has high preclinical and clinical relevance.

For the study of the effect of the antisense oligonucleotides on leukaemias, the animals are grafted with a particular number of cells, and the anti-tumour activity is determined by the increase in the survival time of the treated mice relative to the controls.

To study the effect of the antisense oligonucleotides of the present invention on tumour metastasis, tumour cells are typically treated with the composition ex vivo and then injected into a suitable test animal. The spread of the tumour cells from the site of injection is then monitored over a suitable period of time by standard techniques.

Similar methods can be employed to test the efficacy of the antisense oligonucleotides in combination with chemotherapeutic(s). Suitable controls in this case could include animals treated with the antisense oligonucleotide alone and animals treated with the chemotherapeutic(s) alone.

In vivo toxic effects of the oligonucleotides can be evaluated by measuring their effect on animal body weight during treatment and by performing haematological profiles and liver enzyme analysis after the animal has been sacrificed.

TABLE 3
Examples of xenograft models of human cancer

| Cancer Model | Cell Type |
|---|---|
| Tumour Growth Assay | Mesothelioma (NCI-H2052) |
| Human solid tumour xenografts in | Prostate (PC-3, DU145) |
| mice (sub-cutaneous injection) | Breast (MDA-MB-231, MVB-9) |
| | Colon (HT-29) |
| | Lung (NCI-H460, NCI-H209) |
| | Pancreatic (ASPC-1, SU86.86) |
| | Pancreatic: drug resistant (BxPC-3) |
| | Skin (A2058, C8161) |
| | Cervical (SIHA, HeLa-S3) |
| | Cervical: drug resistant |
| | (HeLa S3-HU-resistance) |
| | Liver (HepG2) |
| | Brain (U87-MG) |
| | Renal (Caki-1, A498) |
| | Ovary (SK-OV-3) |
| Tumour Growth Assay | Breast: drug resistant |
| Human solid tumour isografts in | (MDA-CDDP-S4, MDA-MB435- |
| mice (fat pad injection) | To.1) |
| Survival Assay | Human: Burkitts lymphoma |
| Experimental model of lymphoma | (Non-Hodgkin's) (raji) |
| and leukaemia in mice | Murine: erythroleukemia |
| | (CB7 Friend retrovirus- |
| | induced) |
| Experimental model of lung | Human: melanoma (C8161) |
| metastasis in mice | Murine: fibrosarcoma (R3) |

### 3. Efficacy of Combinations of the Antisense Oligonucleotide and One or More Chemotherapeutics

As indicated above, the antisense oligonucleotides of the invention can be administered in combination with one or more chemotherapeutic agent, for example as a combination product. The efficacy of the combination therapy can be tested in vitro and in vivo as indicated above.

In one embodiment of the present invention, the combination of the antisense oligonucleotide and the chemotherapeutic agent(s) has a greater than additive effect compared to either the antisense oligonucleotide or the chemotherapeutic agent alone. This effect can be measured for example by determining the median lethal dose (LD₅₀) for the chemotherapeutic agent alone and in combination with the antisense oligonucleotide. In another embodiment of the invention, the antisense oligonucleotides increase the cytotoxic effects of one or more chemotherapeutic agents. In a further embodiment, the antisense oligonucleotides act as chemopotentiators of one or more chemotherapeutic agents.

In another embodiment, the combination of the antisense oligonucleotide and the chemotherapeutic agent(s) has an improved therapeutic effect over the therapeutic effect of the antisense oligonucleotide or the chemotherapeutic agent alone. The improved therapeutic effect can be manifested, for example, as an increase in the efficacy of the one or more component of the composition in attenuating tumour growth and/or metastasis and/or a decrease or delay in the toxicity phenomena associated with a component of the combination.

An improved therapeutic effect can be measured, for example, by determining whether the combination results in an improved therapeutic index compared to the individual components.

The ratio of the median effective dose (ED₅₀) and the LD₅₀ can be used as an indication of the therapeutic index of a compound. The ED₅₀ of a drug is the dose required to produce a specified effect in 50% of a test population and the LD₅₀ of a drug is the dose that has a lethal effect on 50% of a test population. The LD₅₀ is determined in preclinical trials, whereas the ED₅₀ can be tested in preclinical or clinical trials. Preclinical trials are conducted using an appropriate animal model, such as those described above. Alternatively the therapeutic index can be determined based on doses that produce a therapeutic effect and doses that produce a toxic effect (e.g. ED₉₀ and LD₁₀, respectively). During clinical studies, the dose, or the concentration (e.g. solution, blood, serum, plasma), of a drug required to produce toxic effects can be compared to the concentration required for the therapeutic effects in the population to evaluate the clinical therapeutic index. Methods of clinical studies to evaluate the clinical therapeutic index are well known to workers skilled in the art.

In one embodiment of the present invention, the antisense oligonucleotide decreases the observed LD₅₀ of at least one of the chemotherapeutic agents in the combination. In another embodiment, the antisense oligonucleotide increases the observed ED₅₀ of at least one of the chemotherapeutic agents in the combination.

An improved therapeutic effect can also be manifested as therapeutic synergy. A combination manifests therapeutic synergy when it is therapeutically superior to one of the components when used at that component's optimum dose [T. H. Corbett et al., (1982) Cancer Treatment Reports, 66, 1187]. To demonstrate the efficacy of a combination, it may be necessary to compare the maximum tolerated dose of the combination with the maximum tolerated dose of each of the separate components in the study in question. This efficacy may be quantified using techniques and equations commonly known to workers skilled in the art. [T. H. Corbett et al., (1977) Cancer, 40, 2660.2680; F. M. Schabel et al., (1979) Cancer Drug Development, Part B, Methods in Cancer Research, 17, 3-51, New York, Academic Press Inc.].

The combination, used at its own maximum tolerated dose, in which each of the components will be present at a dose generally not exceeding its maximum tolerated close, will manifest therapeutic synergy when the efficacy of the combination is greater than the efficacy of the best component when it is administered alone.

In one embodiment of the invention, the combination of the antisense oligonucleotide and a chemotherapeutic agent exhibits therapeutic synergy in the treatment of cancer. In another embodiment, the combination product of the antisense oligonucleotide and a chemotherapeutic agent is efficacious in the treatment of cancer when one or both of the components are used at a less than maximal dose.

### Pharmaceutical Compositions

The antisense oligonucleotide may be administered as a pharmaceutical composition with an appropriate pharmaceutically physiologically acceptable carrier, diluent, excipient or vehicle.

For the treatment of most types of cancel, the pharmaceutical compositions are formulated for systemic administration. For the treatment of mesothelioma, the pharmaceutical compositions can be formulated for intracavitary administration. The term intracavitary includes intraperitoneal, intrapericardial and intrapleural. The pharmaceutical compositions of the present invention may also be formulated for administration orally, topically, parenterally, by inhalation or spray or rectally in dosage unit formulations containing conventional nontoxic pharmaceutically acceptable carriers, adjuvants and vehicles. The term parenteral as used herein includes subcutaneous injections, intravenous, intramuscular, intrasternal injection or infusion techniques.

Aqueous suspensions contain the active compound in admixture with suitable excipients including, for example, suspending agents, such as sodium carboxymethylcellulose, methyl cellulose, hydropropylmethylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

The pharmaceutical compositions may be in the form of a sterile injectable aqueous or oleaginous suspension. This suspension may be formulated according to known art using suitable dispersing or wetting agents and suspending agents such as those mentioned above. The sterile injectable preparation may also be sterile injectable solution or suspension in a non-toxic parentally acceptable diluent or solvent, for example, as a solution in 1,3-butanediol. Acceptable vehicles and solvents that may be employed include, but are not limited to, water, Ringer's solution, lactated Ringer's solution and isotonic sodium chloride solution. Other examples are, sterile, fixed oils which are conventionally employed as a solvent or suspending medium, and a variety of bland fixed oils including, for example, synthetic mono- or diglycerides. In addition, fatty acids such as oleic acid find use in the preparation of injectables.

The pharmaceutical compositions may be in a form suitable for oral use, for example, as tablets, troches, lozenges, aqueous or oily suspensions, dispersible powders or granules, emulsion hard or soft capsules, or syrups or elixirs. Compositions intended for oral use may be prepared according to methods known to the art for the manufacture of pharmaceutical compositions and may contain one or more agents selected from the group of sweetening agents, flavouring agents, colouring agents and preserving agents in order to provide pharmaceutically elegant and palatable preparations. Tablets contain the active ingredient in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed.

Pharmaceutical compositions for oral use may also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Oily suspensions may be formulated by suspending the active ingredients in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may be added to provide palatable oral preparations. These compositions can be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

Pharmaceutical compositions of the invention may also be in the form of oil-in-water emulsions. The oil phase may be a vegetable oil, for example, olive oil or arachis oil, or a mineral oil, for example, liquid paraffin, or it may be a mixtures of these oils. Suitable emulsifying agents may be naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions may also contain sweetening and flavouring agents.

Syrups and elixirs may be formulated with sweetening agents, for example, glycerol, propylene glycol, sorbitol or sucrose. Such formulations may also contain a demulcent, a preservative, and/or flavouring and colouring agents.

Other pharmaceutical compositions and methods of preparing pharmaceutical compositions are known in the art and are described, for example, in “Remington: The Science and Practice of Pharmacy,” Gennaro, A., Lippincott, Williams & Wilkins, Philadelphia, Pa. (2000) (formerly “Remmingtons Pharmaceutical Sciences”).

The amount of active ingredient that is combined with one or more excipients to produce appropriate dosage forms will necessarily vary depending upon the particular component of the combination product, the host treated and the particular route of administration. For example, a formulation intended for oral administration to humans will generally contain, for example, from 0.5 μg to 2 g of active agent compounded with appropriate and convenient amounts of excipients which may vary from about 5 to about 98 percent by weight of the total composition. A formulation intended for parenteral administration to humans will generally contain 0.1 μg to 50 mg. Dosage unit forms will generally contain about 1 μg to about 500 mg of an active ingredient.

### Use of the Antisense Oligonucleotides

The antisense oligonucleotides of the present invention can be used in the treatment of a variety of cancers, including drug resistant tumours. The present invention contemplates that one, or a combination of two or more antisense oligonucleotides may be administered to a patient for the treatment of cancer. The present invention further contemplates that the antisense oligonucleotides can be used in combination with one or more chemotherapeutic agents, i.e. as a combination product, in the treatment of cancer, including drug resistant cancer.

Examples of other cancers which may be may be treated, stabilised, or prevented in accordance with the present invention include, but are not limited to leukaemia, carcinomas, adenocarcinomas, melanomas and sarcomas. Carcinomas, adenocarcinomas and sarcomas are also frequently referred to as “solid tumors,” examples of commonly occurring solid tumors include, but are not limited to, cancer of the brain, breast, cervix, colon, head and neck, kidney, lung, ovary, pancreas, prostate, stomach and uterus, non-small cell lung cancer and colorectal cancer.

The term “leukaemia” refers broadly to progressive, malignant diseases of the blood-forming organs. Leukaemia is typically characterised by a distorted proliferation and development of leukocytes and their precursors in the blood and bone marrow but can also refer to malignant diseases of other blood cells such as erythroleukaemia, which affects immature red blood cells. Leukaemia is generally clinically classified on the basis of (1) the duration and character of the disease—acute or chronic; (2) the type of cell involved—myeloid (myelogenous), lymphoid (lymphogenous) or monocytic, and (3) the increase or non-increase in the number of abnormal cells in the blood—leukaemic or aleukaemic (subleukaemic). Leukaemia includes, for example, acute nonlymphocytic leukaemia, chronic lymphocytic leukaemia, acute granulocytic leukaemia, chronic granulocytic leukaemia, acute promyelocytic leukaemia, adult T-cell leukaemia, aleukaemic leukaemia, aleukocythemic leukaemia, basophylic leukaemia, blast cell leukaemia, bovine leukaemia, chronic myelocytic leukaemia, leukaemia cutis, embryonal leukaemia, eosinophilic leukaemia, Gross' leukaemia, hairy-cell leukaemia, hemoblastic leukaemia, hemocytoblastic leukaemia, histiocytic leukaemia, stem cell leukaemia, acute monocytic leukaemia, leukopenic leukaemia, lymphatic leukaemia, lymphoblastic leukaemia, lymphocytic leukaemia, lymphogenious leukaemia, lymphoid leukaemia, lymphosarcoma cell leukaemia, mast cell leukaemia, megakaryocytic leukaemia, micromyeloblastic leukaemia, monocytic leukaemia, myeloblastic leukaemia, myelocytic leukaemia, myeloid granulocytic leukaemia, myelomonocytic leukaemia, Naegeli leukaemia, plasma cell leukaemia, plasmacytic leukaemia, promyelocytic leukaemia, Rieder cell leukaemia, Schilling's leukaemia, stem cell leukaemia, subleukaemic leukaemia, and undifferentiated cell leukaemia.

The term “sarcoma” generally refers to a tumour which originates in connective tissue, such as muscle, bone, cartilage or fat, and is made up of a substance like embryonic connective tissue and is generally composed of closely packed cells embedded in a fibrillar or homogeneous substance. Sarcomas include soft tissue sarcomas, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumour sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented haemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumour arising from the melanocytic system of the skin and other organs. Melanomas include, for example, acral-lenitiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colorectal carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, haematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, non-small cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “carcinoma” also encompasses adenocarcinomas. Adenocarcinomas are carcinomas that originate in cells that make organs which have glandular (secretory) properties or that originate in cells that line hollow viscera, such as the gastrointestinal tract or bronchial epithelia. Examples include, but are not limited to, adenocarcinomas of the breast, lung, pancreas and prostate. In one embodiment of the invention, the antisense oligonucleotide or combination product is used to treat or stabilise an adenocarcinoma. In another embodiment, the antisense oligonucleotide or combination product is used to treat or stabilise an adenocarcinoma of the lung.

Additional cancers encompassed by the present invention include, for example, Hodgkin's Disease, Non-Hodgkin's lymphoma, multiple myeloma, neuroblastoma, rhabdomyosarcoma, primary thrombocytosis, primary macroglobulinemia, small-cell lung tumors, primary brain tumors, malignant pancreatic insulanoma, malignant carcinoid, urinary bladder cancer, premalignant skin lesions, gliomas, testicular cancer, thyroid cancer, esophageal cancer, genitourinary tract cancer, malignant hypercalcemia, endometrial cancer, adrenal cortical cancer, and medulloblastoma.

In one embodiment of the present invention, the antisense oligonucleotide or combination product is used to treat or stabilise mesothelioma, breast cancel, colorectal cancer, non-small cell lung cancer, stomach cancer, oesophageal cancer, or head and neck cancer, including metastatic and multidrug-resistant versions thereof.

In another embodiment of the invention, the antisense oligonucleotide or combination product is used to treat or stabilise malignant mesothelioma. In the context of the present invention, malignant mesothelioma includes peritoneal mesothelioma, pleural mesothelioma and pericardial mesothelioma. As is known in the art, malignant mesothelioma can be classified as Stage I, II, III or IV. In Stage I mesothelioma (localized malignant mesothelioma) the cancel is found in the lining of the chest cavity near the lung and heart or in the diaphragm or the lung. Stage II, II and IV mesotheliomas are considered to be advanced cancers. In Stage II mesothelioma the cancer has spread beyond the lining of the chest to lymph nodes in the chest. At Stage III the cancer has spread into the chest wall, center of the chest, heart, through the diaphragm, or abdominal lining, and in some cases into nearby lymph nodes and at Stage IV the cancer has spread to distant organs or tissues. Recurrent malignant mesothelioma is a term used to describe mesothelioma that recurs after it has been treated and may recur in the lining of the chest or abdomen or in another part of the body. The present invention contemplates that the antisense oligonucleotides described herein can be used to treat one or more Stages of mesothelioma as well as recurrent mesothelioma and drug resistant mesothelioma.

In a further embodiment, the antisense oligonucleotide or combination product is used to treat or stabilise non-small cell lung carcinoma. Non-small cell lung carcinoma (NSCLC) includes squamous cell carcinoma, adenocarcinoma, and large cell carcinoma. In a specific embodiment, the antisense oligonucleotide or combination product is used to treat or stabilise non-small cell lung cancer adenocarcinoma.

The cancer may be indolent or it may be aggressive. The antisense oligonucleotides can be used to treat refractory cancers, advanced cancers, recurrent cancers and metastatic cancers. One skilled in the art will appreciate that many of these categories may overlap, for example, aggressive cancers are typically also metastatic.

“Aggressive cancer,” as used herein, refers to a rapidly growing cancer. One skilled in the art will appreciate that for some cancers, such as breast cancer or prostate cancer the term “aggressive cancer” will refer to an advanced cancer that has relapsed within approximately the earlier two-thirds of the spectrum of relapse times for a given cancer, whereas for other types of cancer, such as small cell lung carcinoma (SCLC) nearly all cases present rapidly growing cancers which are considered to be aggressive. The term can thus cover a subsection of a certain cancer type or it may encompass all of other cancer types. A “refractory” cancer or tumour refers to a cancer or tumour that has not responded to treatment. “Advanced cancer,” refers to overt disease in a patient, wherein such overt disease is not amenable to cure by local modalities of treatment, such as surgery or radiotherapy. Advanced disease may refer to a locally advanced cancer or it may refer to metastatic cancer. The term “metastatic cancer” refers to cancer that has spread from one part of the body to another.

In one embodiment, the antisense oligonucleotide is conveniently administered to humans by intravenous injection of a sterile aqueous solution at a dose per closing cycle in the range, for example, of 0.1 μg to 1 g, preferably at a dose of 1 mg to 100 mg.

### Combination Therapy

As indicated above, the present invention also contemplates the use of the antisense oligonucleotides in combination with one or more anticancer agents, such as chemotherapeutic agents. The chemotherapeutic agent can be selected from a wide range of cancer chemotherapeutic agents known in the art, including those that target thymidylate synthase. Combination therapies using combinations of standard cancer chemotherapeutics are also known in the art and may be used in conjunction with the antisense oligonucleotides.

Combinations of the antisense oligonucleotide of the present invention and standard chemotherapeutics may act to improve the efficacy of the chemotherapeutic and, therefore, can be used to improve standard cancer therapies. This application is particularly important in the treatment of drug-resistant cancers which are not responsive to standard treatment. Drug-resistant cancers can arise, for example, from heterogeneity of tumour cell populations, alterations in response to chemotherapy and increased malignant potential. Such changes are often more pronounced at advanced stages of disease and have, in part, as an underlying cause, changes in genome/message stability. In one embodiment of the invention, the antisense oligonucleotide of the present invention is used in conjunction with one or more chemotherapeutic agent to treat drug resistant tumours, including drug resistant mesothelioma.

In one embodiment of the invention, the anticancer agent for use with the antisense oligonucleotide belongs to one of three main categories of therapeutic agent:

(i) thymidylate synthase inhibitors such as Tomudex (N-(5-[N-(3,4-dihydro-2-methyl-4-oxoquinazolin-6-ylmethyl)-N-methylamino]-2-thenoyl)-L-glutamic acid) (European Patent Application no. 0239362, Example 7, compound no. 8 therein); Zeneca development compound ZD9331 ((S)-2-(2-fluoro-4-[N-(4-hydroxy-2,7-dimethylquinazolin-6-ylmethyl)-N-(prop-2-ynyl)amino]benzamido)-4-H-1,2,3,4-tetrazol-5-yl) butyric acid) (European Patent Application no. 0562734, Example 3 thereof); LY 231514 (also known as pemetrexed or Alimta®) (Eli Lilly Research Labs, Indianapolis, Ind.); 1843U89 (Glaxo-Wellcome, Research Triangle Park, N.C.); AG337 and AG331 (both by Agouron, La Jolla, Calif.) (Touroutoglou and Pazdur, Clin. Cancer Res., 2, 227-243,1996).

(ii) cytostatic agents such as antioestrogens (for example tamoxifen, toremifene, raloxifene, droloxifene, iodoxyfene), progestogens (for example megestrol acetate), aromatase inhibitors (for example anastrozole, letrazole, vorazole, exemestane), antiprogestogens, antiandrogens (for example flutamide, nilutamide, bicalutamide, cyproterone acetate), LHRH agonists and antagonists (for example goserelin acetate, luprolide), inhibitors of testosterone 5a-dihydroreductase (for example finasteride), anti-invasion agents (for example metalloproteinase inhibitors like marimastat and inhibitors of urokinase plasminogen activator receptor function) and inhibitors of growth factor function, (such growth factors include for example EGF, FGFs, platelet derived growth factor and hepatocyte growth factor such inhibitors include growth factor antibodies, growth factor receptor antibodies, tyrosine kinase inhibitors and serine/threonine kinase inhibitors).

(iii) antiproliferative/antineoplastic drugs and combinations thereof, as used in medical oncology, such as antimetabolites (for example antifolates like methotrexate, fluoropyrimidines like 5-fluorouracil, FUdR, ftorafur, FdUR, purine and adenosine analogues, cytosine arabinoside); antitumour antibiotics (for example anthracyclines like doxorubicin, daunomycin, epirubicin and idarubicin;) mitomycin-C, dactinomycin, mithramycin; platinum derivatives (for example cisplatin, carboplatin, oxaliplatin); alkylating agents (for example nitrogen mustard, melphalan, chlorambucil, busulphan, cyclophosphamide, ifosfamide, nitrosoureas, thiotepa); antimitotic agents (for example vinca alkaloids like vincrisitine and taxoids like taxol, taxotere); topoisomerase inhibitors (for example epipodophyllotoxins like etoposide and teniposide, amsacrine, topotecan).

The anticancer treatment may also be radiotherapy.

In one embodiment of the present invention, the antisense oligonucleotides are used in conjunction with one or more chemotherapeutic agent that targets TS. Examples of suitable TS inhibiting chemotherapeutics include, but are not limited to, the fluoropyrimidine drugs 5-FU, 5-FUdR, capecitabine (an oral form of a pro-drug of 5-FU), ftorafur (also known as Tegafur), Tegafur-uracil (Uftoral® or UFT, a combination of uracil and ftorafur), S-1 (Tegafur combined with 5-chloro-2,4-dihydroxypyridine (CDHP) and potassium oxonate) and a topical 5-FU cream (Effudex®), as well as the non-fluoropyrimidine drugs raltitrexed, methotrexate and pemetrexed (Alimta®). These chemotherapeutic agents are used alone and in combination in a variety of treatment regimens against various cancers including colorectal, breast, lung, stomach, oesophageal, head and neck cancers, and mesothelioma.

In one embodiment of the present invention, the antisense oligonucleotides are used in combination with one or more of 5-FU, 5-FUdR, ftorafur, UFT, S-1, capecitabine, pemetrexed, methotrexate or raltitrexed. In another embodiment, the antisense oligonucleotides are used in combination with 5-FU, 5-FUdR, ftorafur, UFT, S-1, or pemetrexed.

In one embodiment of the invention, the antisense oligonucleotide is used in combination with a chemotherapeutic agent for the treatment of mesothelioma. For the treatment of mesothelioma, the antisense oligonucleotides can be used in combination with doxorubicin, epirubicin, mitomycin, cyclophosphamide, ifosfamide, cisplatin, carboplatin, oxaliplatin, 5-FU, raltitrexed or pemetrexed (Alimta®), or a combination of these chemotherapeutics, such as pemetrexed/cisplatin. In one embodiment of the present invention, the antisense oligonucleotides are used in combination with one or more chemotherapeutics selected from the group of: doxorubicin, epirubicin, mitomycin, cyclophosphamide, ifosfamide, cisplatin, carboplatin, 5-FU, raltitrexed or pemetrexed (Alimta®) in order to potentiate the effect of the chemotherapeutic(s). In a specific embodiment of the invention, the antisense oligonucleotide is used in combination with pemetrexed for the treatment of mesothelioma.

In one embodiment of the invention, the antisense oligonucleotide is used in combination with a chemotherapeutic agent for the treatment of non-small cell lung carcinoma (NSCLC). For the treatment of NSCLC, the antisense oligonucleotides can be used in combination with camptothecin, carboplatin (paraplatin), cisplatin (platinol), oxaliplatin, epirubicin, gemcitabine, irinotecan (CPT-11), navelbine (vinorelbine), oxaliplatin, pemetrexed (Alimta®), taxol (paclitaxel) or taxotere (docetaxel). Pemetrexed is typically used as second line therapy for NSCLC for patients who have failed first line treatment. Pemetrexed in combination with cisplatin has, however, recently shown efficacy as a first line treatment of NSCLC, in particular in a subgroup with adenocarcinoma histology.

In a specific embodiment of the invention, the antisense oligonucleotide is used in combination with pemetrexed for the treatment of NSCLC. The combination may further comprise cisplatin and may be used as a first or second line therapy.

5-FU has been used as chemotherapeutic for many years alone and in conjunction with other chemotherapeutics. The following exemplary therapeutic regimens are provided with the understanding that one skilled in the art would appreciate that they may be applied to the situations where 5-FU is used alone or conjunction with another chemotherapeutic. A first exemplary regimen is the Mayo regimen, wherein 1 cycle consists of 5-FU administered at 425 mg/m² by intravenous bolus injection daily together with 20 mg/m² leucovorin for 5 days, followed by 3 weeks off. A second therapeutic regimen may consist of administering 200 to 220 mg/m². 5-FU by continuous infusion over 24 hours once a week. A third therapeutic regimen consists of shorter, intermittent infusions of 5-FU from between 24 to 120 hours, every week, two weeks, three weeks or four weeks at dosages of 600 mg/m² to 2500 mg/m² per 24 hours. One skilled in the art will also appreciate that 5-FU and its variants can be used in combination therapies with a variety of other traditional chemotherapeutic drugs.

An exemplary therapeutic regimen for raltitrexed (Tomudex®) is administration at 3 mg/m² once every 3 weeks by bolus injection.

An exemplary regimen for pemetrexed is, administration at 500 mg/m² once every 3 weeks. Pemetrexed may be used in this regimen alone or in combination with cisplatin. Examples of additional supportive drugs that could be included in the above regimen include: folic acid daily at about 0.4 mg, Vitamin B12 at 1000 micrograms every 9 weeks. Dexamethazone may also be included as a supportive drug.

Other chemotherapeutic agents contemplated by the present invention include those which may be applicable to a range of cancers, such as doxorubicin, capecitabine, mitoxantrone, irinotecan (CPT-11), as well as those that are suited to the treatment of a specific cancer.

Examples of chemotherapeutic agents suitable for the treatment of breast cancer include, but are not limited to, capecitabine, cyclophosphamide, ifosfamide, cisplatin, carboplatin, 5-fluorouracil (5-FU), taxol, taxanes such as paclitaxel and docetaxel and various anthracyclines, such as doxorubicin and epi-doxorubicin (also known as epirubicin). Combination therapies using standard cancer chemotherapeutics may also be used in conjunction with the antisense oligonucleotides and are also well known in the art, for example, the combination of epirubicin with paclitaxel or docetaxel, or the combination of doxorubicin or epirubicin with cyclophosphamide, which are used for breast cancer treatments. Polychemotherapeutic regimens are also useful and may consist, for example, of doxorubicin/cyclophosphamide/5-fluorouracil or cyclophosphamide/epirubicin/5-fluorouracil. Many of the above chemotherapeutics and combinations thereof are useful in the treatment of a variety of solid turnouts.

Cyclophosphamide, mitoxantrone and estramustine are known to be suitable for the treatment of prostate cancer. Cyclophosphamide, vincristine, doxorubicin and etoposide are used in the treatment of small cell lung cancer, as are combinations of etoposide with either cisplatin or carboplatin. In the treatment of stomach or oesophageal cancer, combinations of doxorubicin or epirubicin with cisplatin and 5-fluorouracil are useful. For colorectal cancer, CPT-11 alone or in combination with 5-fluorouracil-based drugs, or oxaliplatin alone or in combination with 5-fluorouracil-based drugs can be used. Oxaliplatin may also be used in combination with capecitabine.

Other examples include the combination of cyclophosphamide, doxorubicin, vincristine and prednisone in the treatment of non-Hodgkin's lymphoma; the combination of doxorubicin, bleomycin, vinblastine and DTIC in the treatment of Hodgkin's disease and the combination of cisplatin or carboplatin with any one or a combination of gemcitabine, paclitaxel, docetaxel, vinorelbine or etoposide in the treatment of non-small cell lung cancer. Pemetrexed alone is also a proven effective drug in the treatment of non-small cell lung cancer. Other suitable chemotherapeutic agents include, but are not limited to, mitomycin C, vinblastine, IL-2, novantrone, DTIC and hydroxyurea.

The combination product of the invention may be used as a first line therapy in the treatment of cancer, or it may be used as a second line therapy for patients who have failed first line therapy.

One embodiment of the present invention contemplates the use of the antisense oligonucleotides as “sensitizing agents,” or “chemopotentiators,” which selectively inhibit the growth of cancer cells. In this case, the antisense oligonucleotides alone does not have a cytotoxic effect on the cancer cell, but provides a means of weakening the cancer cells, and thereby facilitates the benefit from conventional anti-cancer therapeutics.

In one embodiment of the invention, there is provided a combination comprising a TS targeted antisense oligonucleotide and a TS targeting chemotherapeutic that demonstrates therapeutic synergy in the treatment of cancer. Such combinations can be used to provide greater efficacy of treatment in a subject than treatment with one component alone. Alternatively, as such combinations remain effective when one or both of the components of the combination is used at a less than maximal close, the combination can be used with one or both of the components at sub-maximal dose to provide efficacious treatment while decreasing side-effects, such as toxicity. Accordingly, in one embodiment of the invention, there is provided a combination product comprising a TS targeted antisense oligonucleotide and a TS targeting chemotherapeutic, in which the amounts of the individual components can be adjusted to provide an optimal therapeutic index (ratio of efficacy to toxicity) for the patient to be treated. The optimal therapeutic index can be based on the patient's priorities with respect to toxicity and survival, thus allowing for an individualized approach to treatment. Accordingly, in one embodiment of the invention, the combination product comprises maximal doses (for example, at or close to the MTD) of the antisense oligonucleotide and the TS targeting drug and is useful for maximizing efficacy in patients. In another embodiment, the combination product comprises less than maximal doses of the antisense oligonucleotide and the TS targeting drug and is useful for lowering the toxicity of the treatment while still providing sufficient efficacy.

### Clinical Trials in Cancer Patients

One skilled in the art will appreciate that, for the treatment of human patients, the antisense oligonucleotides, alone or in combination with one or more chemotherapeutic agents, should be tested in Clinical Trials in order to further evaluate their efficacy in the treatment of cancer and to obtain regulatory approval for therapeutic use. As is known in the art, clinical trials progress through phases of testing, which are identified as Phases I, II, III, and IV.

Initially the antisense oligonucleotides will be evaluated in a Phase I trial. Typically Phase I trials are used to determine the best mode of administration (for example, by pill or by injection), the frequency of administration, and the toxicity for the compounds. Phase I studies frequently include laboratory tests, such as blood tests and biopsies, to evaluate the effects of a compound in the body of the patient. For a Phase I trial, a small group of cancer patients are treated with a specific dose of the antisense oligonucleotide(s). During the trial, the dose is typically increased group by group in order to determine the maximum tolerated dose (MTD) and the dose-limiting toxicities (DLT) associated with the compound. This process determines an appropriate dose to use in a subsequent Phase II trial.

A Phase II trial can be conducted to evaluate further the effectiveness and safety of the antisense oligonucleotides. In Phase II trials, the antisense oligonucleotide is administered to groups of patients with either one specific type of cancer or with related cancers, using the dosage found to be effective in Phase I trials.

Phase III trials focus on determining how a compound compares to the standard, or most widely accepted, treatment. In Phase III trials, patients are randomly assigned to one of two or more “arms”. In a trial with two alms, for example, one arm will receive the standard treatment (control group) and the other arm will receive treatment with the antisense oligonucleotide (investigational group).

Phase IV trials are used to further evaluate the long-term safety and effectiveness of a compound. Phase IV trials are less common than Phase I, II and III trials and will take place after the antisense oligonucleotide has been approved for standard use.

### Eligibility of Patients for Clinical Trials

Participant eligibility criteria can range from general (for example, age, sex, type of cancer) to specific (for example, type and number of prior treatments, tumour characteristics, blood cell counts, organ function). Eligibility criteria may also vary with trial phase. For example, in Phase I and II trials, the criteria often exclude patients who may be at risk from the investigational treatment because of abnormal organ function or other factors. In Phase II and III trials additional criteria are often included regarding disease type and stage, and number and type of prior treatments.

Phase I cancer trials usually comprise 15 to 30 participants for whom other treatment options have not been effective. Phase II trials typically comprise up to 100 participants who have already received chemotherapy, surgery, or radiation treatment, but for whom the treatment has not been effective. Participation in Phase II trials is often restricted based on the previous treatment received. Phase III trials usually comprise hundreds to thousands of participants. This large number of participants is necessary in order to determine whether there are true differences between the effectiveness of the antisense oligonucleotides and the standard treatment. Phase III may comprise patients ranging from those newly diagnosed with cancer to those with extensive disease in order to cover the disease continuum.

One skilled in the art will appreciate that clinical trials should be designed to be as inclusive as possible without making the study population too diverse to determine whether the treatment might be as effective on a more narrowly defined population. The more diverse the population included in the trial, the more applicable the results could be to the general population, particularly in Phase III trials. Selection of appropriate participants in each phase of clinical trial is considered to be within the ordinary skills of a worker in the art.

### Assessment of Patients Prior to Treatment

Prior to commencement of the study, several measures known in the art can be used to first classify the patients. Patients can first be assessed, for example, using the Eastern Cooperative Oncology Group (ECOG) Performance Status (PS) scale or the Karnofsky Performance Status (KPS) scale, both of which are widely accepted standards for the assessment of the progression of a patient's disease as measured by functional impairment in the patient.

Patients' overall quality of life can be assessed, for example, using the McGill Quality of Life Questionnaire (MQOL) (Cohen et al (1995) Palliative Medicine 9: 207-219). The MQOL measures physical symptoms; physical, psychological and existential well-being; support; and overall quality of life. To assess symptoms such as nausea, mood, appetite, insomnia, mobility and fatigue the Symptom Distress Scale (SDS) developed by McCorkle and Young ((1978) Cancer Nursing 1: 373-378) can be used.

Patients can also be classified according to the type and/or stage of their disease and/or by tumour size.

### Administration of the Antisense Oligonucleotides of the Present Invention in Clinical Trials

The antisense oligonucleotide is typically administered to the trial participants parenterally. In one embodiment, the antisense oligonucleotide is administered by intravenous infusion. Methods of administering drugs by intravenous infusion are known in the art. Usually intravenous infusion takes place over a certain time period, for example, over the course of 60 minutes. In other embodiments of the invention, for example, for the treatment of patients with mesothelioma, the antisense oligonucleotide is administered intracavitorially, i.e. by intrapleural, intraperitoneal or intrapericardial infusion.

### Monitoring of Patient Outcome

The endpoint of a clinical trial is a measurable outcome that indicates the effectiveness of a treatment under evaluation. The endpoint is established prior to the commencement of the trial and will vary depending on the type and phase of the clinical trial. Examples of endpoints include, for example, tumour response rate—the proportion of trial participants whose tumour was reduced in size by a specific amount, usually described as a percentage; disease-free survival—the amount of time a participant survives without cancer occurring or recurring, usually measured in months; overall survival—the amount of time a participant lives, typically measured from the beginning of the clinical trial until the time of death. For advanced and/or metastatic cancers, disease stabilisation—the proportion of trial participants whose disease has stabilised, for example, whose tumour(s) has ceased to grow and/or metastasise, can be used as an endpoint. Other endpoints include toxicity and quality of life.

Tumour response rate is a typical endpoint in Phase II trials. However, even if a treatment reduces the size of a participant's tumour and lengthens the period of disease-free survival, it may not lengthen overall survival. In such a case, side effects and failure to extend overall survival might outweigh the benefit of longer disease-free survival. Alternatively, the participant's improved quality of life during the tumour-free interval might outweigh other factors. Thus, because tumour response rates are often temporary and may not translate into long-term survival benefits for the participant, response rate is a reasonable measure of a treatment's effectiveness in a Phase II trial, whereas participant survival and quality of life are typically used as endpoints in a Phase III trial.

### Pharmaceutical Kits

The present invention additionally provides for therapeutic kits containing the antisense oligonucleotide or combination product as described above for use in the treatment of cancer. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. In this case the container means may itself be an inhalant, syringe, pipette, eye dropper, or other such like apparatus, from which the composition may be administered to a patient.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, syringe, pipette, forceps, measured spoon, eye dropper or any such medically approved delivery vehicle.

To gain a better understanding of the invention described herein, the following examples are set forth. It should be understood that these examples are for illustrative purposes only. Therefore, they should not limit the scope of this invention in any way.

### EXAMPLES

### Example 1

### Example 1.1

### Experimental Methods

Cell culture: MCF-7 (human breast adenocarcinoma) cells were cultured in DMEM supplemented with 10% fetal bovine serum (FBS), 2 mM glutamine, 10 mM Hepes (pH 7.4) and 0.1% Gentamycin.

Vector construction: Expression vectors pAS/TSS and pAS/exon1,2 were designed to produce, upon transfection into MCF-7 cells, single-stranded antisense RNA molecules containing double-stranded 30 bp oligonucleotides complementary to the TS mRNA at one of two sites. Oligodeoxynucleotides corresponding to each strand of the human TS cDNA at positions 111 to 140 (pAS/TSS; targeting a 30 bp region adjacent to, and 2 bp away from, the translation start site) or 296 to 325 (pAS/exon1, 2; targeting a 30 bp region spanning the exon 1/exon 2 boundary) were synthesized. Numbering of bases was according to GenBank accession no. X02308 (Takeishi et al., 1985, Nucleic Acids Res. 13: 2035-2043). In order to facilitate cloning, six additional nucleotides were incorporated (5 at the 5′ end, 1 at the 3′ end) of each oligodeoxynucleotide to produce Hind III or Xba I sticky ends when complementary strands were annealed. Single-stranded oligonucleotides (4 mg each) were annealed in 3×SET (450 mM NaCl, 60 mM Tris-HCl, 3 mM EDTA, pH 7.8) by treating the mixture for 5 mill at 90° C., 5 h at 50° C., then 16 h at 25° C. Double-stranded products were identified by gel electrophoresis, and directionally inserted into the Hind III and Xba I sites of pRC/CMV (Invitrogen Corp., San Diego, Calif.). The orientation of cloning was confirmed by direct sequencing.

Oligodeoxynucleotides: Fully phosphorothioated 20-mer oligonucleotides (ODNs) were synthesized by Isis Pharmaceuticals (Carlsbad, Calif.). The 6 terminal nucleotides at the 5′ and 3′ ends of each ODN were 2′-methoxyethoxy-modified to make them resistant to intracellular nucleases and increase their stability within cells (McKay et al., 1996, Nucleic Acids. Res. 24: 411-417), but the internal 8 nucleotides without methoxyethoxy groups were susceptible to RNase H cleavage. Oligo 86 was complementary to TS mRNA from base positions 1035 to 1054 (GenBank accession no. X02308; Takeishi et al., 1985, ibid), which surround the TS mRNA translation stop site (UAG at bases 1045 to 1047). Oligo 90 was complementary to base positions 111 to 130, which are 3′ and proximal to the translation start site (AUG at bases 106 to 109). Oligo 92 was complementary to base positions. 101 to 120, including and surrounding the translation start site.

Transfection: Antisense RNA expression vectors were transfected into MCF-7 cells using Lipofectamine (GIBCO BRL, Burlington, ON, Canada), a polycationic liposome formulation. 1.5×10⁶ cells were dispensed into 100×15 mill tissue culture plates and allowed to adhere overnight. Cells were washed once with 3 ml OptiMEM (GIBCO BRL, Burlington, ON, Canada), followed by exposure to 6 ml of a mixture of double-stranded expression vector DNA (0.83 μg/ml) plus Lipofectamine (1.67 μg/ml) in opti-MEM for 6 h at 37° C. in a 5% CO₂ incubator. The DNA/Lipofectamine mixture was removed and replaced with 10 ml DMEM with supplements. Control cells were transfected with pRC/CMV vector without insert. Transiently-transfected cells were used within 1 to 6 days. Stably-transfected cells were allowed to recover in complete nonselective medium without Geneticin for 48 h, then grown in the presence of Geneticin (400 μg per ml, active form [Gibco/BRL]) to allow selection of colonies harboring pRC/CMV control vectors, or pRC/CMV vectors expressing RNA complementary to sequences surrounding the translation start site (pAS/TSS).

Single-stranded oligodeoxynucleotides (0.5 μM or 1.0 μM) were transiently transfected into 1.5×10⁶ MCF-7 cells adhering to 100×15 mm tissue culture plates, in a total volume of 5 ml in the presence of Lipofectamine (2 μg/ml) in Opti-MEM. Control cells were treated with Lipofectamine/Opti-MEM without added DNA. Cells were washed after 6 h, medium plus supplements added as described above, and then grown for 48 hours before isolation of nuclei for run-on transcription measurement.

Southern blot analysis: Total DNA was isolated from pAS/TSS transfected cells as follows. Cells were incubated in lysis buffer (50 mM Tris-HCl [pH8.0], 100 mM EDTA, 100 mM NaCl, 1% sodium dodecyl sulfate, 0.5 mg/ml proteinase K) for 6 h at 55° C. One-third the volume of 6 M NaCl was added to precipitate non-nucleic acids by centrifugation at 10,000×g for 15 min. The DNA in the supernatant was precipitated in isopropanol and washed with 70% ethanol. DNA was cleaved with Hind III for 16 h and analysed by Southern blotting (Sambrook et al., 1989, Molecular Cloning: A Laboratory Manual. Cold Spring Harbor, N.Y.: Cold Spring Harbor Laboratory). Blots were hybridized with an [α-³²P]dCTP random primer-labeled pAS/TSS probe (Church and Gilbert, 1984, Proc. Natl. Acad. Sci. USA 81:1991-1995), and were exposed to a phosphor screen and quantitated using a PhosphorImager and the ImageQuant program (Molecular Dynamics, Sunnyvale, Calif.). The nylon membranes were stripped and rehybridized with an Alu probe (300 bp of a human Alu restriction fragment inserted into pBR322 [Jelinek et al., 1980, Proc. Natl. Acad. Sci. USA 77:1398-1402]) in order to quantitate the amount of human DNA loaded in each lane (Koropatnick et al., 1988, Mol. Biol. Med. 5:69-83).

Northern blot analysis: RNA was isolated using RNeasy columns (Qiagen Inc., Chatsworth, Calif.) from cells transfected with the pAS/TSS expression vector. Ten or 15 μg of RNA per lane were separated on a 1.4% formaldehyde gel (Sambrook et al., 1989, ibid) and transferred to a Hybond-N nylon membrane. Membranes were hybridized (Church and Gilbert, 1984, ibid) with either a pAS/TSS-generated riboprobe (Promega Corp., Madison, Wis.) designed to bind to antisense RNA, or a random primer-labeled, 1.9 kb Xho I fragment from pcHTS-1 (a eukaryotic expression vector containing the human TS cDNA: a generous gift from Dr. K. Takeishi, University of Shizuoka, Shizuoka, Japan) (Takeishi et al., 1985, Nucleic Acids Res. 13:2035-2043). Blots were stripped and rehybridized with cDNA probes to detect 18S ribosomal RNA or glyceraldehyde-3-phosphate dehydrogenase (GAPDH). Images were quantitated using a PhosphorImager (Molecular Dynamics, Sunnyvale, Calif.).

Isolation of nuclei: Relative transcription rates were determined by a nuclear run-on assay (Koropatnick et al., 1997, BioTechniques 22:64-66), a modification of the methods of Kikuchi et al. (1992, J. Biol. Chem. 267: 21505-21511) and Almendral et al. (1988, Mol. Cell. Biol. 8: 2140-2148). Nascent transcripts were extended in vitro (Marzluff and Huang, 1984, IRL Press, Oxford, 89-129) in parallel reactions using MCF-7 cell nuclei isolated 48 h following transient transfection with control or antisense TS RNA expression vectors, or single-stranded antisense TS oligodeoxynucleotides (or Lipofectamine alone), and from cells stably transfected with antisense RNA expression vectors. Adherent cells were rinsed twice with ice-cold PBS, scraped off with a rubber policeman, pelleted in PBS (5 min, 500×g) and lysed by incubating 5 min at 4° C. in 4 ml of lysis buffer (10 mM Tris-Cl, pH 7.4, 10 mM NaCl. 3 mM MgCl2, 0.50% NP-40). All subsequent steps were carried out at 4° C. Complete cell lysis and integrity of released nuclei was checked by light microscopy, and nuclei were pelleted by centrifugation at 500×g for 5 min. Nuclei were then resuspended in 4 ml of lysis buffer by vortexing, pelleted by centrifugation, resuspended in 200 μl of nuclei storage buffer (40% glycerol, 5 mM MgCl₂, 50 mM Tris-HCl [pH 8.0], 0.1 mM EDTA) in a 15 ml conical polypropylene centrifuge tube, and immediately frozen in liquid nitrogen and stored at −120° C. until use up to one month later.

Run-on transcription: RNA elongation reactions were performed for 30 min at 30° C. using 2×10⁷ nuclei/400 μl reaction. Reaction mixtures were composed of 200 μl nuclei storage buffer plus 200 μl of sterile 2× reaction buffer (10 mM Tris-HCl [pH 8.0], 5 mM MgCl2, 0.3 M KCl, 1 mM ATP, 1 mM CTP, 1 mM GTP, 5 mM dithiothreitol, and 2 μl [α-³²P]UTP or [[α-³²P]CTP [˜3000 Ci/mmole, 10 mCi/ml]). Nucleotides, radionucleotides, and dithiothreitol were added immediately prior to use. Nascent RNA transcripts were allowed to elongate for 30 min at 30° C. on a shaking platform, followed by addition of 600 μl of RNase-free DNase I (0.04 units RQ1 DNase I [RNase-free; Promega Corp.], 0.5 M NaCl, 50 mM MgCl₂, 2 mM CaCl₂, 10 mM Tris-HCl [pH 7.4]). The ³²P-labeled RNA was isolated using Trizol (Gibco/BRL), and the final precipitated RNA was dissolved in Church hybridization buffer (1 mM EDTA, 0.5 M NaHPO₄ [pH 7.2], 7% sodium lauryl sulphate [SLS]), to a final concentration of 4×10⁶ cpm per ml.

Hybridization of radiolabeled RNA to immobilized unlabeled probes: In order to distinguish between TS sense and antisense RNA molecules produced in isolated nuclei from transfected cells, target DNA (immobilized on nitrocellulose filters in triplicate dots, 2 μg per dot) consisted of single-stranded synthetic oligonucleotides rather than TS cDNA. Strand-specific oligonucleotide probes were also used to assess levels of human metallothionein-2 (MT-2) mRNA and antisense RNA as positive and negative controls, respectively. Single-stranded oligonucleotides were immobilized on nitrocellulose filters by dissolving in 6×SSC (16 μg per ml) and applying 125 μl per dot using a BioRad dot-blot apparatus. Unlabeled complementary cDNA probes for GAPDH mRNA (Denhardt et al., 1988, Oncogene 2:55-59) and 18S ribosomal RNA (Behrend et al., 1994, Cancer Res. 54:832-837) were denatured and immobilized on the same nitrocellulose filters (5 μg per dot, triplicate dots) using a previously-described protocol (Koropatnick, 1988, Proc. Soc. Exp. Biol. Med. 188:287-300). Hybridization of radiolabeled RNA to these dots assessed transcription of GAPDH and 18S rRNA genes, and acted as internal standards against which to measure changes in TS gene transcription. For cells transfected with single-stranded oligonucleotides, TS gene transcription was assessed by hybridization of radiolabeled TS RNA transcripts to immobilized target DNA consisting of a 1.9 kb Xhol fragment isolated from pCHTS-1.

Nitrocellulose filters containing triplicate clots of oligonucleotide and cDNA probes to assess run-on transcription of antisense TS RNA expression vectors, and endogenous TS, MT-2, GAPDH and 18S rRNA genes, were prehybridized in Church buffer for 20 mill at 65° C. in a Hybrid hybridization chamber. The prehybridization buffer was then removed, 2 mls of radiolabeled RNA resulting from 30 min of run-on transcription in isolated nuclei (in Church hybridization buffer, 4×10⁶ (cpm per ml) was added, and the filters were hybridized for 48 h at 65° C. The filters were then washed twice at 65° C. in posthybridization buffer (40 mM Na2HPO₄, 1% SDS; 20 min per wash). Posthybridization buffer was removed and 8 ml of RNase A (1 μg per ml in 6×SSC) was added and incubated for 30 min at 37° C. to reduce signal from unhybridized radiolabeled RNA. After a final wash in posthybridization buffer (10 min, 37° C.) filters were blotted dry and bound radioactivity visualized and quantitated using a phosphorimager and the ImageQuant program (Molecular Dynamics, Sunnyvale, Calif.). Relative transcription of antisense TS expression vectors, endogenous TS genes, and MT-2 genes was defined as:

$$Relative transcription rate = \frac{( hybridisation signal from gene of interest )}{( hybridisation signal for GAPDH or 18 S rRNA genes )}$$

TS oligonucleotide probes: Bases in bold-face (below) form part of restriction endonuclease sites, and are not sense or antisense TS sequences. Numbering indicates the distance from the beginning of the transcription start site.

TS cDNA Nucleotides 111 to 140:

sense TS (JK-5):

[SEQ ID NO:23]

CTAGATGTGGCCGGCTCGGAGCTGCCGCGCCGGCCA
antisense TS (JK-2):

[SEQ ID NO:24]

AGCTTGGCCGGCGCGGCAGCTCCGAGCCGGCCACAT

TS cDNA Nucleotides 296 to 325:

sense TS (JK-3):

[SEQ ID NO:25]

CTAGAGCTACAGCCTGAGAGATGAATTCCCTCTGCA
antisense TS (JK-4).

[SEQ ID NO:26]

AGCTTGCAGAGGGAATTCATCTCTCAGGCTGTAGCT

MT-2 oligonucleotide probes (Karin and Richards, 1982, Nucleic Acids Res. 10: 3165-3173): Sense and antisense oligonucleotide sequences did not have non-complementary sequences added to the 5′ and 3′ ends. Numbering indicates the distance from the translation start site.

MT cDNA Nucleotides −14 to 6:

| | |
|---|---|
| sense MT: CTCTTCAGCACGCCATGGAT | [SEQ ID NO:27] |

MT cDNA Nucleotides 204 to 223:

| | |
|---|---|
| antisense MT: AGGGTCTACCTTTCTTGCGC | [SEQ ID NO:28] |

### Example 1.2

### Antisense Oligodeoxynucleotide Targeting Regions at or Near the Translation Stop Site at the 3′ End of the TS Gene as a Method to Inhibit Growth of Human Tumour Cells

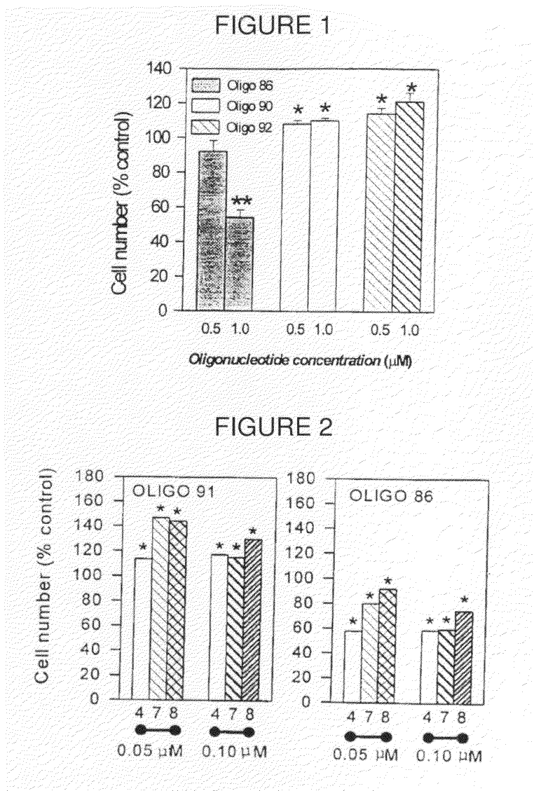

a) A 20-mer antisense oligodeoxynucleotide (oligo 86) targeted to the translation stop site at the 3′ end of the thymidylate synthase mRNA is growth inhibitory (cytostatic) in a human breast cancer cell line (MCF-7 cells). Antisense oligonucleotides of the same length (oligos 90 and 92), targeted to regions at or near the translation start site at the 5′ end of the TS mRNA, are not cytostatic (FIG. 1).

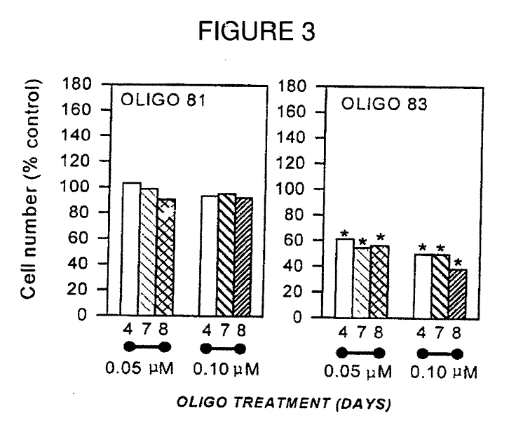

b) A 20-mer antisense oligodeoxynucleotides targeted to the TS mRNA translation start site (oligos 91 and 93) did not inhibit growth of a human cervical carcinoma (HeLa) cell line. In fact, growth was significantly enhanced. Antisense oligodeoxynucleotides targeted to the 3′ end of the TS mRNA, including the translation stop site (oligo 86) or a sequence in the 3′ untranslated region (oligo 83) significantly inhibited HeLa cell growth. An antisense TS oligonucleotide targeted to another sequence in the 3′ untranslated region of TS mRNA (oligo 81) had no effect on HeLa cell growth (FIGS. 2 and 3).

Therefore, no antisense TS oligodeoxynucleotides targeted to the translation start site was successful in inhibiting growth of two different human tumour cell lines (human breast carcinoma MCF-7 cells or human cervical carcinoma HeLa cells). Two separate antisense TS oligodeoxynucleotides targeted to the 3′ end of the TS gene were potent inhibitors of human tumour cell growth.

### Example 1.3

(a) Antisense oligodeoxynucleotide targeting of the thymidylate synthase translation stop site as a method to enhance human tumour cell sensitivity to the toxic effects of Tomudex (ZD 1694).

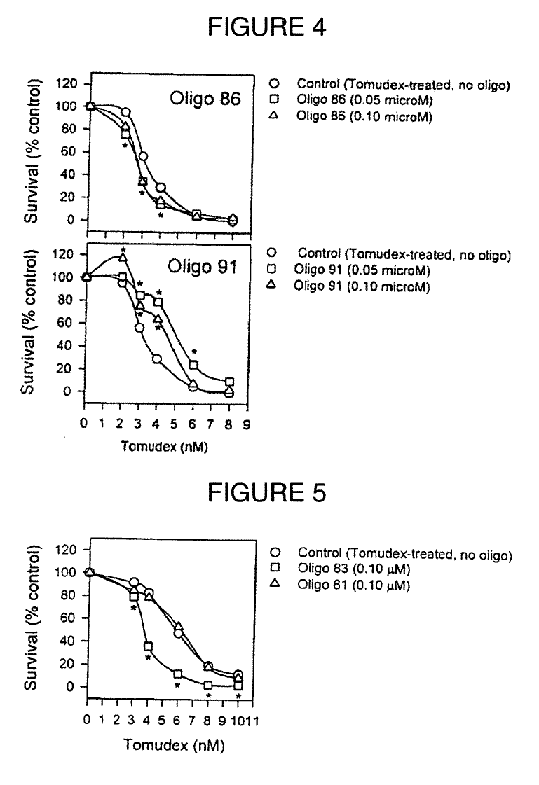

Antisense TS oligodeoxynucleotides (oligos 86 and 83) targeting sequences in the 3′ untranslated region of TS mRNA enhanced human cervical carcinoma cell sensitivity to Tomudex. The enhancement in sensitivity was in addition to the directly cytostatic effects of oligos 86 and 83 (FIGS. 4 and 5).

(b) Antisense oligodeoxynucleotide targeting of the thymidylate synthase translation start site as a method to enhance human cell resistance to the toxic effects of Tomudex (ZD 1694).

An antisense TS oligodeoxynucleotide (oligo 91) targeting the translation start site of TS mRNA enhanced human cervical carcinoma cell resistance to the toxic effects of Tomudex (FIG. 4).

### Example 1.4

### Induction of Transcription of Genes Targeted with Antisense Nucleic Acids as a Screening Method to Identify Appropriate Target Sequences for Antisense Nucleic Acids

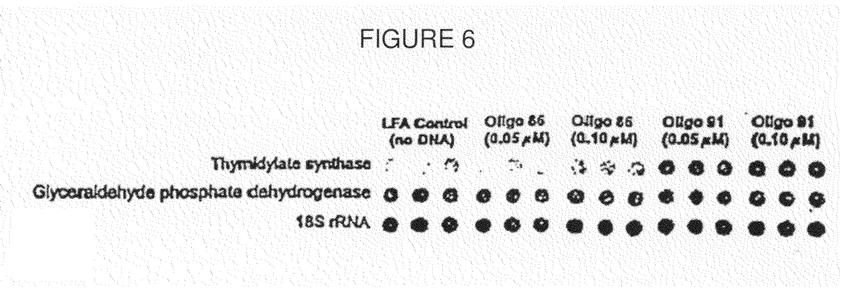

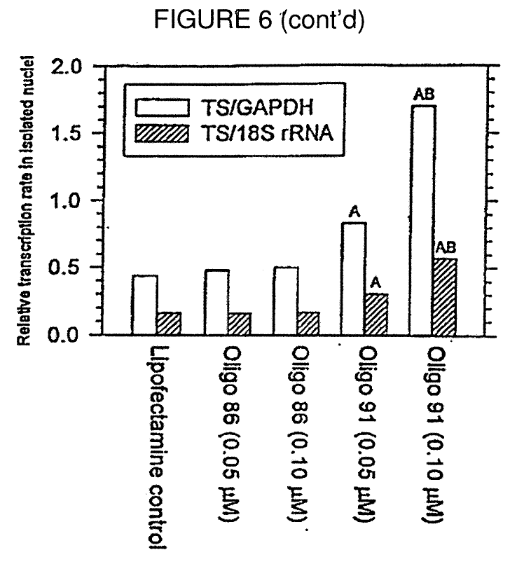

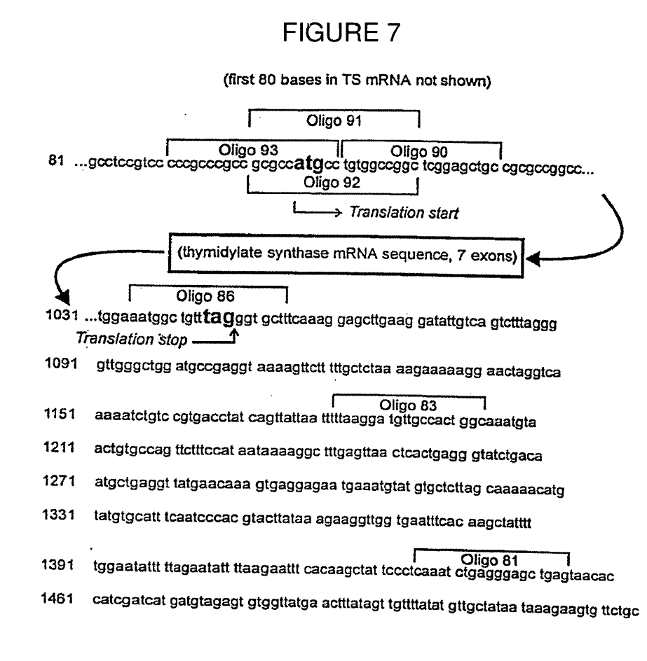

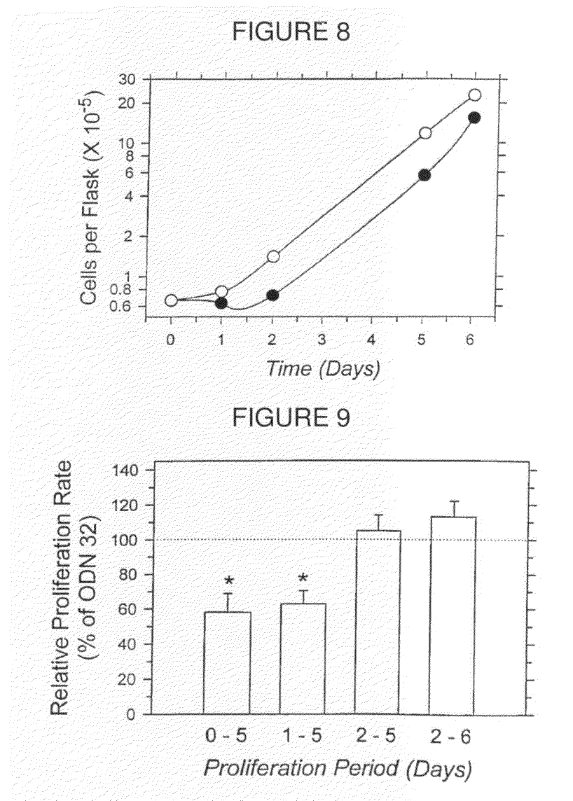

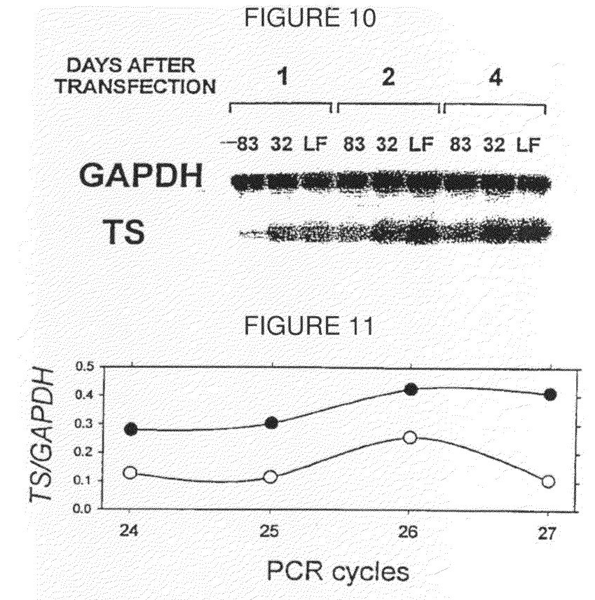

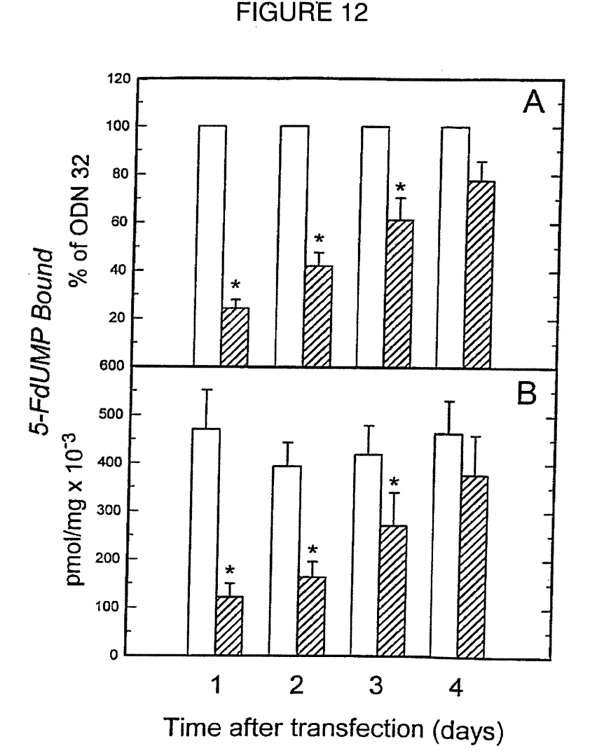

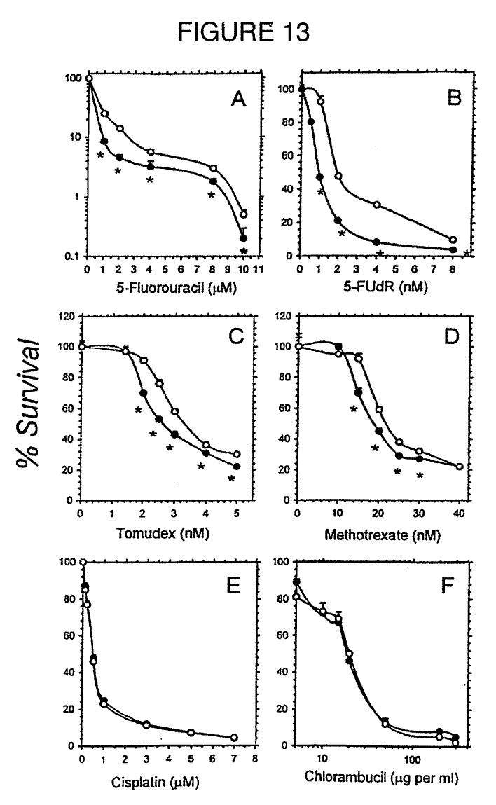

Human tumour cells appear to compensate for antisense inactivation of specific mRNA by increasing transcription of genes producing the target sequences, a process that can be termed “compensatory transcription,” resulting in resistance to the effectiveness of antisense nucleic acids. It has been observed, that TS gene transcription is induced in human MCF-7 breast carcinoma cells by treatment with antisense TS RNA and oligodeoxynucleotides targeted to regions at or near the TS mRNA translation start site. The same phenomenon has been observed in human HeLa cells transiently-transfected with antisense TS oligo 91 (targeted to the translation start site), but not in response to oligo 86 (targeted to the translation stop site) (FIG. 6). Increased specific gene transcription in response to transfected antisense nucleic acids would indicate that the target sequence is inappropriate to achieve downregulated gene expression. On the other hand, it may be an appropriate sequence to target to achieve upregulated gene expression (to increase resistance to chemotherapeutic drugs, for example, in normal tissues).

In summary, the above examples have demonstrated that antisense oligonucleotides, targeted against selected regions of thymidylate synthase mRNA, can effectively inhibit growth when administered alone. They can also enhance cell killing by Tomudex. Conversely, antisense oligonucleotides targeted to certain mRNA regions (for example, the translation start site) may either be ineffective, or enhance growth and survival during exposure to Tomudex. Ineffectiveness may be due to oligonucleotide-induced TS gene transcription. It is essential to identify TS mRNA regions that may be effectively targeted with antisense sequences to inhibit tumour cell growth and enhance the toxicity of anticancer drugs. Furthermore, the mechanism by which antisense sequences targeted to 5′ TS mRNA regions induce TS gene transcription has important implications for choosing antisense targets in TS mRNA in particular, and for optimising antisense strategies in general.

### Example 2

### Example 2.1

### Experimental Methods

Oligodeoxynucleotides: Fully phosphorothioated 20-base oligonucleotides were synthesised by ISIS Pharmaceuticals (Carlsbad, Calif., USA). The 6 nucleotides on either end of the oligomer were methoxyethoxylated in the 2′-position, enhancing hybridisation as well as resistance to exonucleation. The middle 8 nucleotides were not methoxyethoxylated to allow RNase H endonucleation and degradation of mRNA hybridised to the oligomer. ODN 83 is complementary to TS mRNA, starting from a position 136 bases downstream of the translational stop site (5′-GCCAGTGGCAACATCCTTAA-3′ [SEQ ID NO:1]). ODN 32 is a randomised sequence of ODN 83 (5′-ATGCGCCAACGGTTCCTAAA-3′ [SEQ ID NO:7]), with the same base constituents in random order. A search of available mRNA sequences using the NCBI BLAST search tool revealed that ODN 83 had sequences of 10 or more complementary bases to only human TS mRNA, while ODN 32 had sequences of 10 or more complementary bases to no known mRNAs.

Radioisotope: [6-³H]5-FUdR (specific activity 18.6 Ci/mmol) was purchased from Moravek Biochemicals (Brea, Calif., USA). This isotope was 99.98% pure upon initial production, with a degradation rate of 0.5-1% per month at −20° C., and was used within 3 months of manufacture.

Other supplies: Cell culture chemicals and nutrients were obtained from Canadian Life Technologies (GIBCO) (Burlington, Ontario, Canada). All other chemicals were obtained from commercial sources. Plasticware was purchased from VWR Canlab (Mississauga, Ontario, Canada) and Fisher Scientific Uniondale, Ontario, Canada).

Cell Culture: Human cervical carcinoma HeLa cells were maintained in D-MEM plus 10% foetal bovine serum and penicillin (50 units/ml)/streptomycin (50 μg/ml). Cultures were incubated in a humidified atmosphere of 5% CO₂ at 37° C. Rapidly proliferating cells were utilised for establishing cultures of experimental cells, which were allowed to plate overnight prior to manipulation.

Transfection was performed using lipofectamine (LFA, GIBCO-BRL), a polycationic liposome formulation. Cells to be used for proliferation experiments were plated at a starting cell number of between 0.6 and 1×10⁵ cells per 25-cm tissue culture flask, and LFA was used at 3 μg/ml. For cells in 75-cm flasks, which were to be harvested and extracted for assay of mRNA or TS content, the starting cell number was approximately 8−10×10⁵, and the LFA concentration was 4 μg/ml. Prior to transfection, adherent HeLa cells were washed once with PBS and then treated with antisense or scrambled control ODN (50 nM) in the appropriate concentration of LFA in serum-free D-MEM, at 37° C. for 6.0 h. The cells were then washed once with PBS and cultured in the presence of D-MEM plus 10% FBS. In cells treated with cytotoxic agents, exposure was initiated 24 hours after the removal of LFA/ODN, by addition of 0.2-volume of growth medium containing the agent at 6 times the final concentration. At the time of addition of drug, and after 4 days of incubation, cell numbers were determined from replicate flasks by enumerating with a particle counter (Coulter Electronics, Hialeah, Fla., USA). The proliferation of drug treated cells (fold-increase in cell number) was calculated as a percentage of that of the control cells. IC₅₀ and IC₉₀ values were determined by interpolation of plotted data.

RT-PCR to measure TS mRNA: RNA was isolated from transfected cells using Trizol (GIBCO-BRL). Complementary DNA was synthesised from 1 μg of total RNA using 200 U of Moloney Murine Leukemia Virus reverse transcriptase (GIBCO BRL) in 50 mM Tris-HCl (pH 8.3), 75 mM KCl, 3 mM MgCl₂, 1 mM mixed dNTPs, 100 pmol random primers and 10 mM dithiothreitol at 37° C. for 1 hr. The enzyme was inactivated at 95° C. for 5 min. The resulting cDNAs (in a volume of 2.5 μl) were amplified in a polymerase chain reaction (PCR) using 1.25 U of Taq DNA polymerase in 50 μl 20 mM Tris-HCl pH 8.4), 50 mM KCl, 0.2 mM mixed dNTPs, 2 mM MgCl₂, and 50 pmol of primers specific for TS and GAPDH cDNAs. TS and GAPDH cDNAs were amplified together in the same reaction tube to allow the level of housekeeping GAPDH cDNA to be used to determine the relative level of TS mRNA. Twenty-four to 27 cycles of PCR amplification (94° C. 45 s, 55° C. 30 s, 72° C. 90 s) produced fragments of 208 bp and 752 bp using primer sets for TS (forward 5′-CACACTTTGGGAGATGCACA-3′ [SEQ ID NO:29]; reverse 5′-CTTTGAAAGCACCCTAAACAGCCAT-3′ [SEQ ID NO:30]) and GAPDH (forward 5′-TATTGGGCGCCTGGTCACCA-3′ [SEQ ID NO:31]; reverse 5′-CCACCTTCTTGATGTCATCA-3′ [SEQ. ID NO:32]), respectively. PCR products were separated on a 1.2% agarose gel, and transferred to Hybond nylon membrane: (Amersham, Canada, Ltd., Oakville, Ontario, Canada) by Southern blotting.

Blots were hybridised to [α-³²P]dCTP random primer-labeled probe (pcHTS-1, a generous gift from Dr. K. Takeishi, University of Shizuoka, Shizuoka, Japan; or a cDNA insert recognising glyceraldehyde-3-phosphate dehydrogenase [GAPDH]). Hybridisation signals were quantified using a PhosphorImager and ImageQuant (Molecular Dynamics, Sunnyvale, Calif., USA).

TS binding assay: Cellular content of TS was assayed by binding of [6-³H]5-FdUMP. This method was demonstrated to label total TS unless the cells were pretreated with 5-FU or 5-FUdR. The assay was performed using cells that were treated with antisense ODN 83 or the scrambled control ODN 32. Briefly, cells were harvested by scraping into PBS and resuspending the subsequent pellet in 100 mM KH₂PO₄ (pH 7.4). Cells were disrupted by freezing and thawing, followed by sonication. The total protein concentration was determined using Coomassie staining (BioRad reagent) (MI) in order to express results as pmol 5-FdUMP bound per mg total protein. 5-FdUMP binding was assessed in paired lysates from cells transfected with ODN 83 or ODN 32, in separate incubation reactions carried out on different days; however, pairs were always assessed together under the same reaction conditions. On each occasion, the incubation vessel contained 50 μg of total protein, 75 μM methylene-FH4, 100 mM mercaptoethanol, 50 mM KH₂PO₄ (pH 7.4), and 15 nM [6-³H]5-FdUMP in a final volume of 200 μl. After 30 min at 37° C., the incubation vas stopped by addition of 5 volumes of albumin-coated, activated charcoal. After 10 min (room temperature), this slurry was centrifuged (3000×g, 30 min, 22° C.), and the supernatant re-centrifuged to completely remove particulate matter. Two aliquots of 300 μl each were removed from the final, clarified supernatant for scintillation counting.

Statistical analysis: Data for cell growth after treatment with ODNs alone, or in combination with cytotoxic drugs, are presented as the mean +/−standard error or standard deviation as determined by Student t-test. For determinations of FdUMP binding, differences between paired samples from cells transfected with different ODNs were assessed using a paired t-test. This controlled for differences in experimental conditions on each of the 5 occasions that FdUMP binding was assessed. In all cases, significance was chosen a priori to be indicated by differences at a confidence level of p<0.02.

### Examples 3-10

### Antisense Oligonucleotides

The antisense oligonucleotide referred to throughout Examples 3-10 as SEQ ID NO:1 or ODN 83 is a phosphorothioated ODN with 2′-methoxy-ethoxy modification on the 6 nucleotides at both the 5′- and 3′-ends, unless otherwise indicated.

The scrambled control ODN (SEQ ID NO:7): 5′-ATGCGCCAACGGTTCCTAAA-3′ has the same base composition as ODN SEQ ID NO:1, in random order, and is not complementary to any region of human TS mRNA and is referred to in the preceding Examples as “Oligo 32” or “ODN 32”.

### Example 3

### Effect of TS Antisense Oligonucleotide Treatment on Tumour Cell Proliferation

HeLa, MCF-7, HT-29, MSTO-211H, NCI-H2052, and NCI-H28 cell lines were plated at a density of 100,000 cells/flask (T-25) and were treated the following clay with ODNs (10, 25, 50 or 100 nM) using LipofectAmine 2000 (Invitrogen) as at transfection agent. Cells were counted using a Coulter electronic particle counter after four days. Cell proliferation in the presence of TS antisense SEQ ID NO:1 is shown relative to proliferation in control ODN (SEQ ID NO:7) treated flasks.

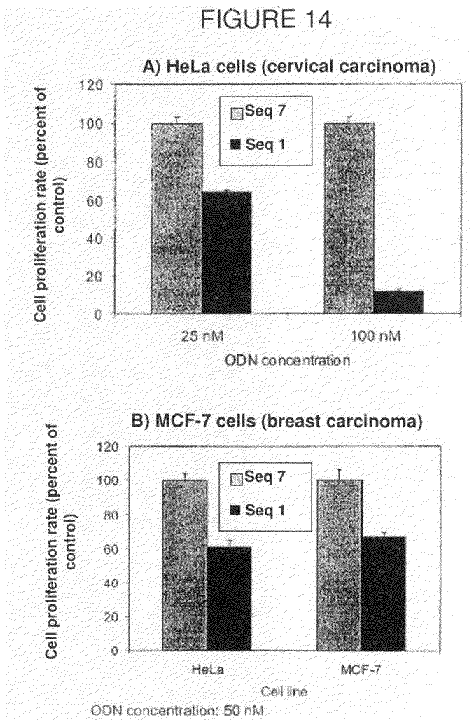

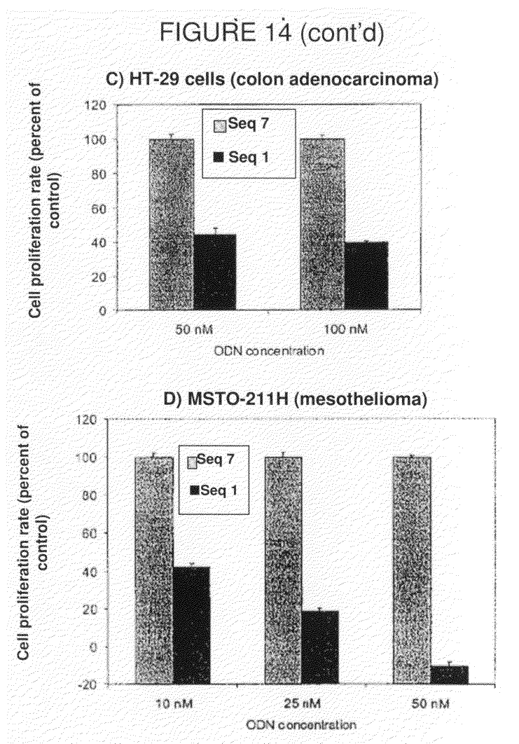

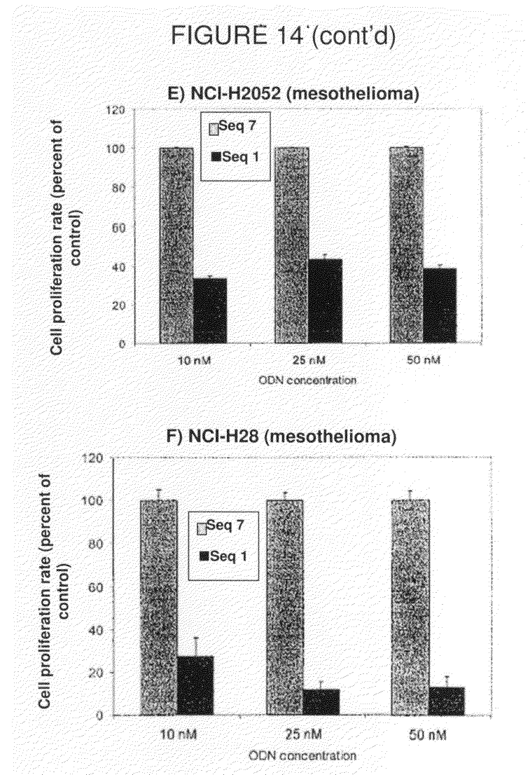

FIG. 14 shows that three mesothelioma-derived cell lines, NCI-H28, MSTO-211H and NCI-H2052, are exquisitely sensitive to TS antisense ODN treatment. Inhibition of proliferation of the breast tumour-derived MCF-7 and ovarian carcinoma OV-90 cells by TS antisense SEQ ID NO: 1 was also observed.

### Example 4

### Effect of TS Antisense Oligonucleotide Treatment on TS mRNA Levels in Tumour Cells

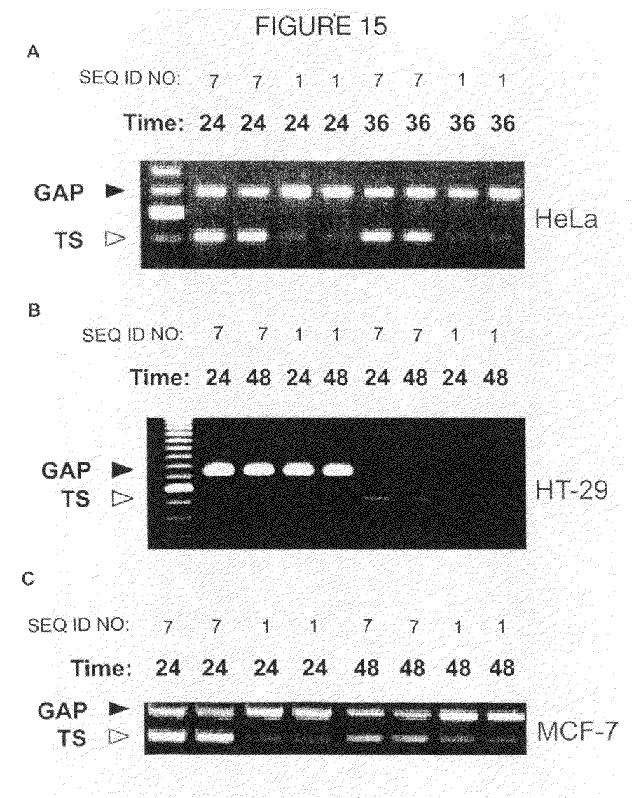

HeLa, HT-29 and MCF-7 cells were plated in T75 flasks at a density of 10⁶ cells per flask. After attachment, the cells were treated with TS antisense SEQ ID NO: 1 or scrambled control SEQ ID NO:7 (100 nM) mixed with LipofectAmine 2000 (1 μg/ml). After 24, 36, or 48 hours (as indicated), RNA was extracted using Trizol (Invitrogen). The RNA was reverse-transcribed and cDNA used as a template for PCR using specific primers for glyceraldehyde 3-phosphate dehydrogenase (GAPDH) or TS, as previously described (Berg et al. (2001) J Pharmacol Exp Ther 298:477-484; Berg et al. (2003) Cancer Gene Therapy 10:278-286). The abundance of GAPDH mRNA was unchanged by ODN treatment, while levels of TS mRNA in each cell line were specifically reduced by SEQ ID NO: 1 treatment relative to control SEQ ID NO:7 treatment (FIG. 15).

### Example 5

### Antisense ODN Delivery into Tumour Cell Lines as Monitored by In Situ Hybridization

An in situ hybridization assay to quantitate the effectiveness of antisense ODN delivery to tumour cells was developed. Human HeLa, MCF7 and HT-29 tumour cells were transfected with TS antisense ODNs using cationic lipid and fixed at various times with paraformaldehyde. In situ hybridization of biotinylated complementary ODNs was detected using streptavidin-horseradish peroxidase followed by diaminobenzidine.

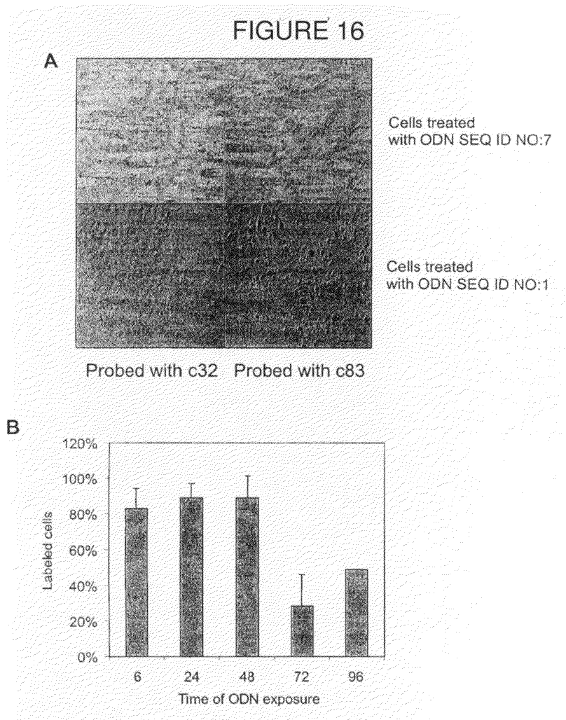

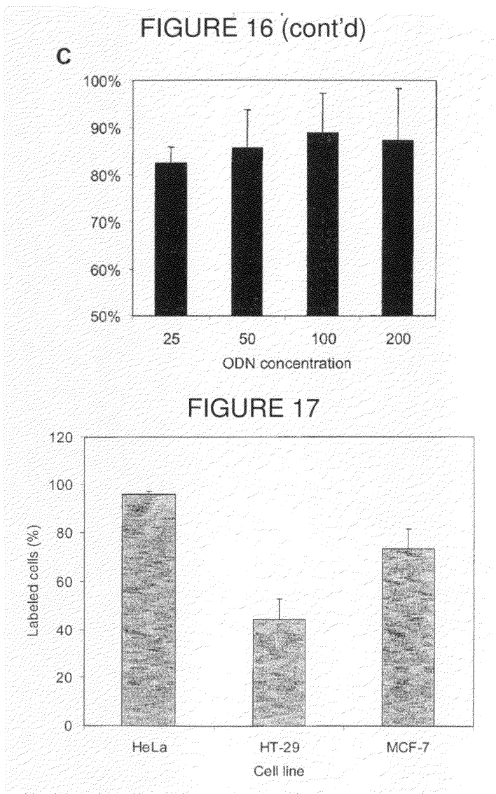

In more detail, HeLa cells were treated with TS antisense SEQ ID NO:1 or scrambled control SEQ ID NO:7 in the presence of LipofectAmine 2000 (1 μg/ml) and fixed with paraformaldehyde. Biotin-labelled complementary ODNs (c83 and c32) were used as in situ hybridization probes, detected with horseradish peroxidase-conjugated avidin and DAB as a substrate. Intense staining suggests mainly nuclear localisation of internalised ODNs (FIG. 16A). Quantitation of stained cells are shown in FIG. 16 for HeLa cells treated with 100 nM ODN for various times (FIG. 16B) or treated for 24 hours with various ODN concentrations (FIG. 16C). Following treatment for 6, 24, 36 and 48 hours with ODN (25, 50 or 100 nM), 83 to 90% of HeLa cells were intensely labelled. In accord with previous results showing recovery of TS mRNA and protein levels at later times, only 29% of cells fixed 72 hours after ODN exposure were stained.

ODN delivery into tumour cells was compared in HeLa, HT-29, and MCF-7 tumour cell lines. Uptake of antisense ODN into MCF-7 breast carcinoma cells was comparable to HeLa cells, and RT-PCR analysis indicated similar reductions in TS mRNA levels in these two cell lines. In contrast, only 46% of HT-29 colon carcinoma cells were labelled using the in situ hybridization technique, and ODN-mediated TS mRNA downregulation and ODN-induced inhibition of cell proliferation were also less effective in HT-29 compared with HeLa cells.

### Example 6

### Effect of Treatment with TS Antisense ODN in Combination with TS-Targeting Chemotherapeutic Agents on HT-29 Cells

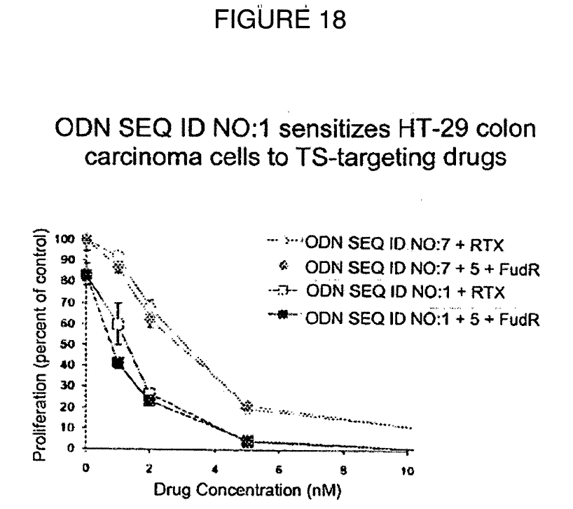

HT-29 cells were plated in T25 flasks, treated with SEQ ID NO: 1 or SEQ ID NO:7 (50 nM) and various concentrations of raltitrexed (RTX) or 5-fluorouracil deoxyribonucleotide (5-FudR). Cell proliferation was measured by counting cells after 4 days. The results shown in FIG. 18 indicate increased inhibition of HT-29 proliferation by a combination of SEQ ID NO:1 and chemotherapeutic compared to control SEQ ID NO:7 and chemotherapeutic.

### Example 7

### Effect of TS Antisense ODN Treatment on Tumour Cell Proliferation

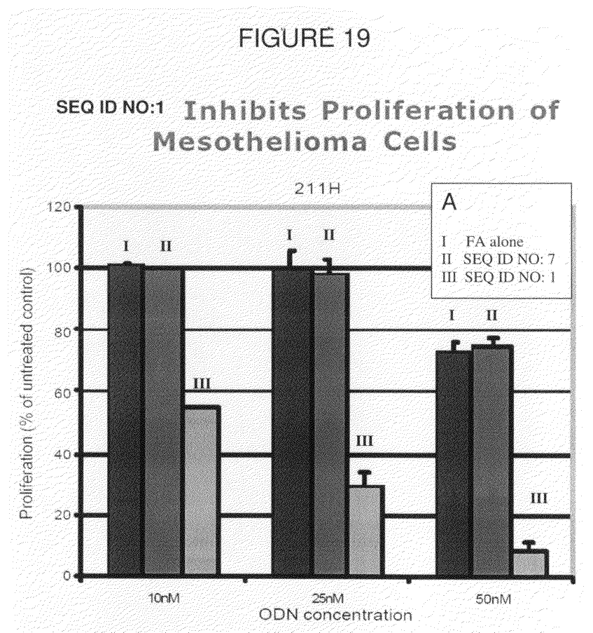

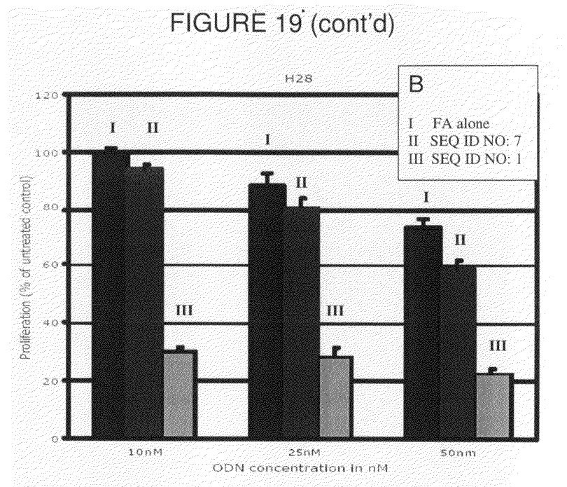

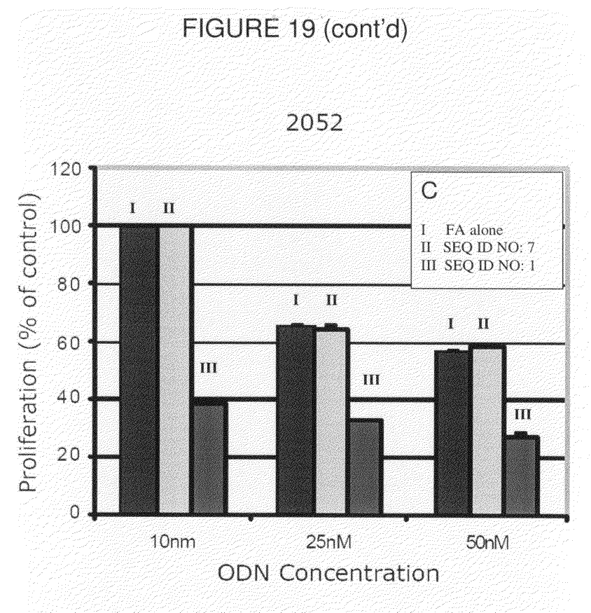

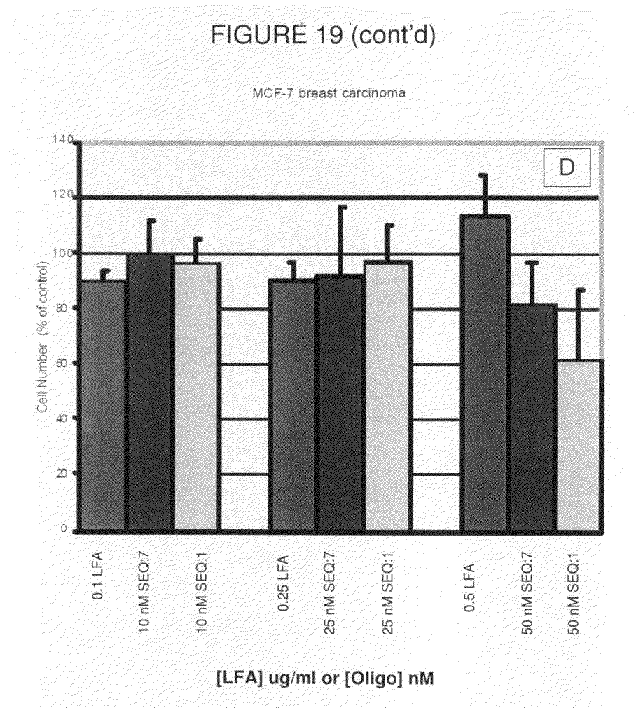

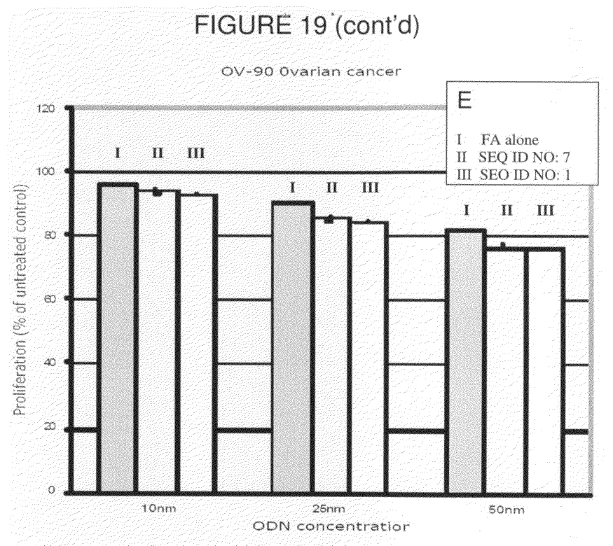

To measure the effects of ODNs on tumour cell proliferation, MSTO-211H, NCI-H28, NCI-H2052, MCF-7, and OV-90 cells were plated at a density of 1×10⁵ per 25-cm² flask. After 24 or 48 hours, ODNs were mixed with Lipofectamine 2000 (LFA, Invitrogen) for 15 minutes in serum-free medium. Additional medium and FBS were added to achieve the final concentrations of ODN indicated in FIG. 19. Final concentrations of LFA ranged from 0.1 to 0.5 μg/ml to maintain a constant ODN:lipid ratio. The cell culture medium was replaced with 2 ml of the ODN:lipid mixture, and cells incubated for 4 hours, after which an additional 2 ml of medium with FBS was added. Cells in 3 flasks were counted using an electronic particle counter (Beckman Coulter, Hialeah, Fla.) at the time of treatment and after three days of culture. Cell proliferation in the presence of TS antisense SEQ ID NO: 1 or control ODN (SEQ ID NO:7) is shown relative to proliferation in the presence of LFA alone.

FIG. 19 shows that three mesothelioma-derived cell lines, NCI-H28, MSTO-211H and NCI-H2052, are exquisitely sensitive to TS antisense ODN (SEQ ID NO:1) treatment. Inhibition of proliferation of the breast tumour-derived MCF-7 and ovarian carcinoma OV-90 cells by TS antisense ODN SEQ ID NO: 1 was also observed.

### Example 8

### Effect of TS Antisense Oligonucleotide Treatment on TS mRNA Levels in Mesothelioma Cells

Mesothelioma cells were treated with TS antisense ODN SEQ ID NO:1 or control ODN SEQ ID NO:7 (10, 25 and 50 ng) mixed with LFA as described in Example 7. After 24 hours, RNA was isolated using Trizol (Invitrogen). RNA (2 ug) was reverse-transcribed using SuperScript II. Two to 5% of the resulting cDNA was amplified by PCR using primers specific for GAPDH or TS. PCR products were separated on a 1.75% agarose gel stained with ethidium bromide. Image Master VDS gel documentation system and Image Quant software were used to quantitate staining intensity.

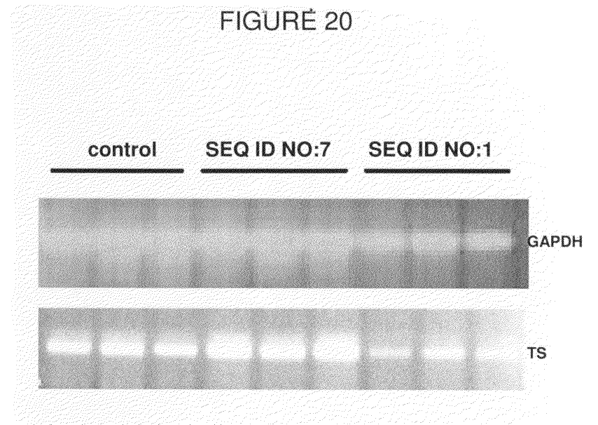

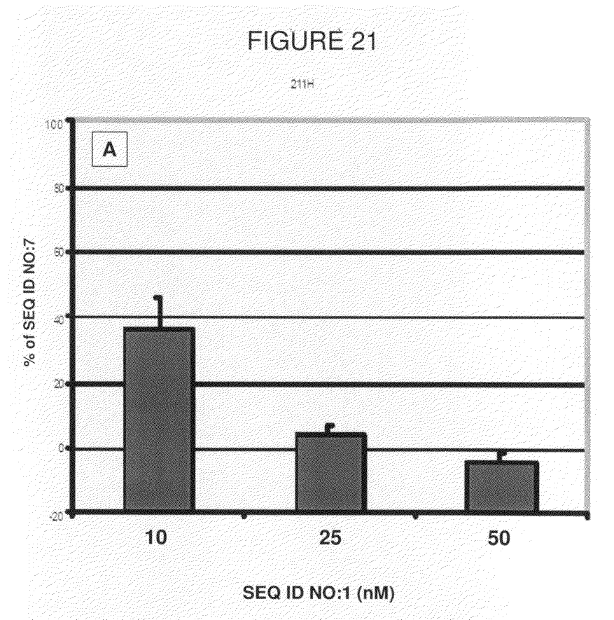

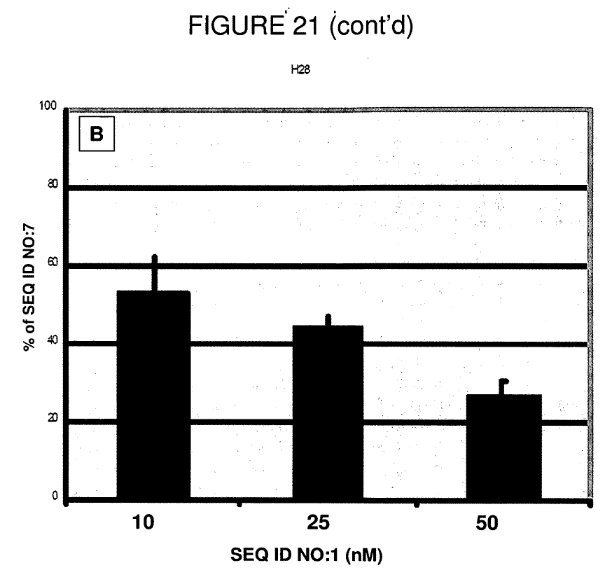

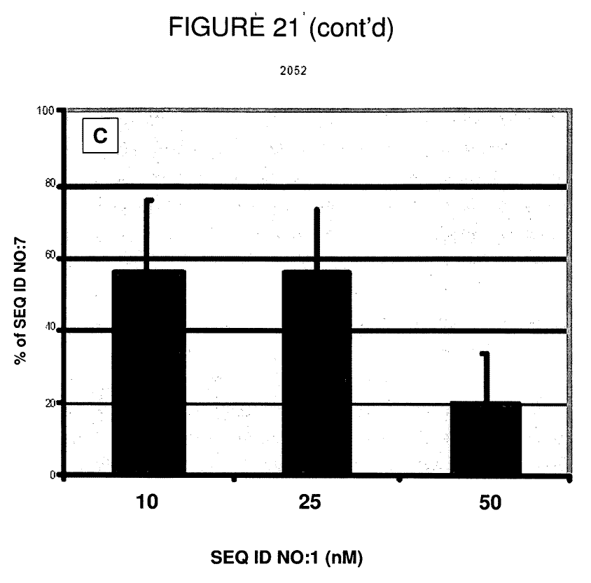

A representative example of RT-PCR products from 3 replicates of mesothelioma 211H cells treated with TS antisense ODN SEQ ID NO: 1 or control ODN SEQ ID NO:7 is shown in FIG. 20. FIG. 21A-C illustrates the quantitation of RT-PCR products from mesothelioma 211H, 2052, and H28 cells treated with TS antisense ODN SEQ ID NO:1 compared to control ODN SEQ ID NO:7 relative to GAPDH PCR products. The mean plus standard deviation (n=3) is shown. FIGS. 20 and 21 show that the levels of TS mRNA in each cell line were specifically reduced by SEQ ID NO:1 treatment relative to control SEQ ID NO:7 treatment.

### Example 9

### Effect of Treatment with TS Antisense ODN in Combination with TS-Targeting Chemotherapeutic Agents on Mesothelioma Cell Lines

Tumour cells were first treated with pemetrexed (Alimta®), 5-FUdR, or gemcitabine alone at various concentrations to determine the IC₅₀ in a proliferation assay, using cell counting on day 0 and day 3.

Mesothelioma and OV-90 cells were pretreated with TS antisense ODN SEQ ID NO:1 plus control ODN SEQ ID NO:7 (5 nM each) or control ODN SEQ ID NO:7 (10 nM) with 0.1 μg/ml LFA for 4 hours as described in Example 7. MCF-7 cells were pretreated with 25 nM SEQ ID NO:7 or SEQ ID NO: 1 (mixed with 0.25 ug/ml LFA). After the 4-hour incubation, 2 ml of medium with various concentrations of Alimta®, 5-FUdR or gemcitabine was added to achieve the final drug concentrations indicated. Cells were counted at the time of drug treatment and 3 days later, as described in Example 7.

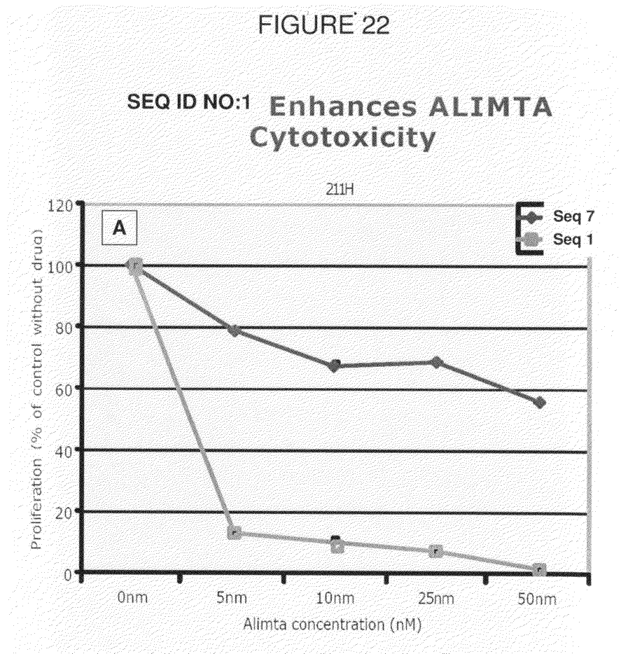

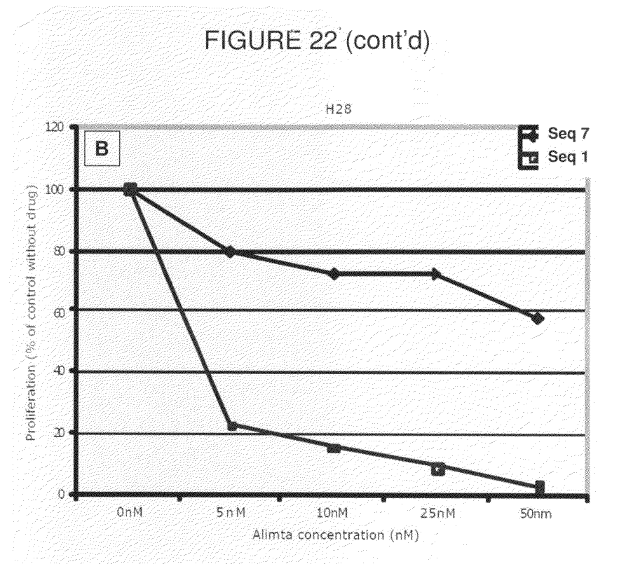

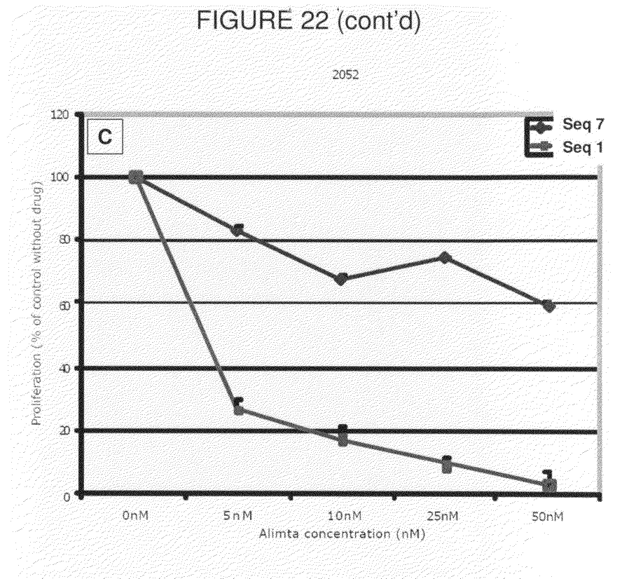

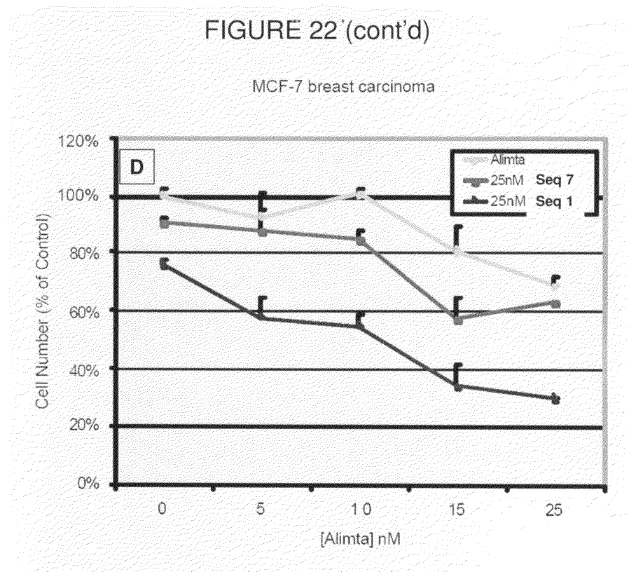

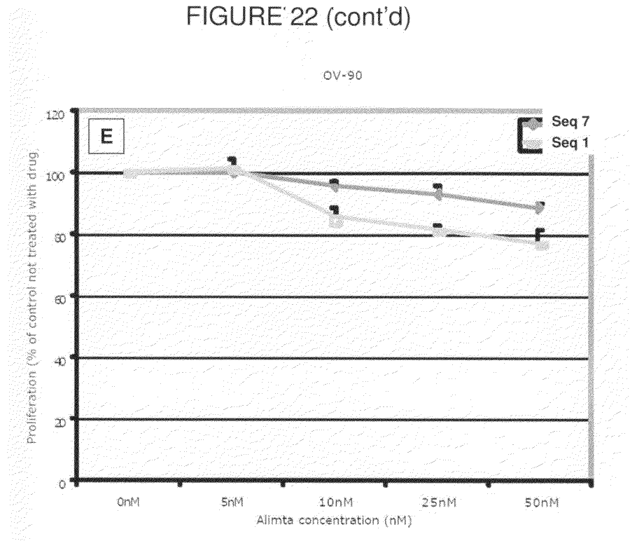

FIG. 22 illustrates the effect of TS antisense ODN SEQ ID NO:1 in combination with Alimta® on tumour cell proliferation. The mean IC₅₀ for Alimta® in the three mesothelioma cell lines was 22.5 nM (+/−4.3 nM). Combined treatment with TS antisense ODN (5 nM) reduced the Alimta® IC₅₀ by fourfold to 5 nM (+/−2.5 nM) in a greater than additive fashion and thus potentiates Alimta® cytotoxicity. Control scrambled ODN SEQ ID NO:7 had no effect on the IC₅₀, alone or in combination with Alimta®. The ability of TS antisense ODN SEQ ID NO:1 to potentiate the cytotoxic effect of Alimta® in MCF-7 cells and OV-90 cells was not as pronounced.

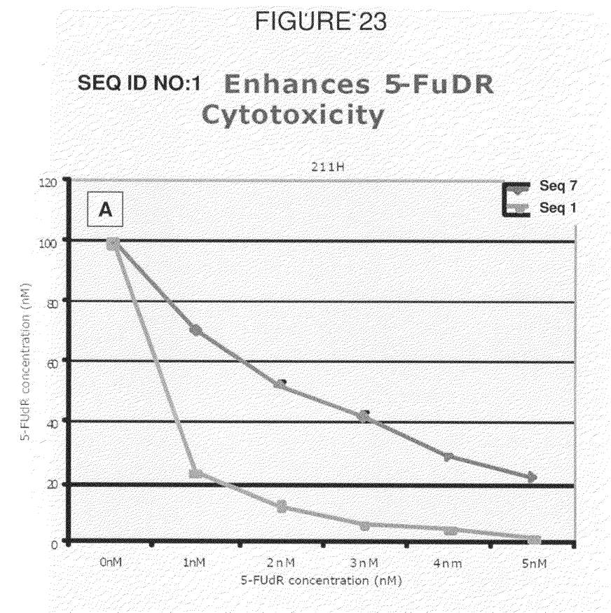

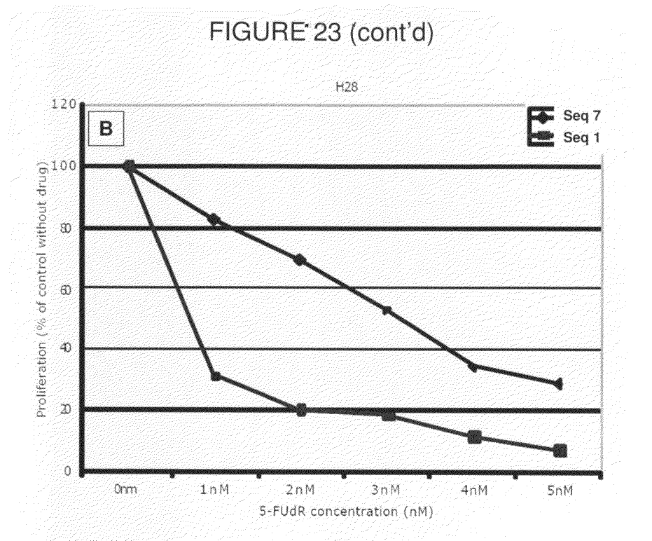

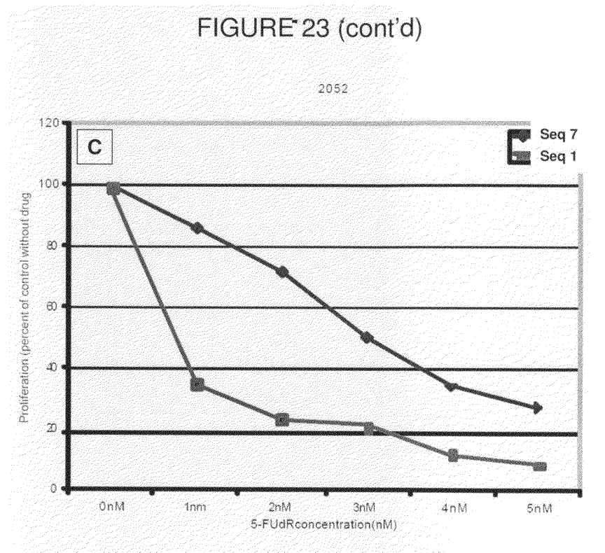

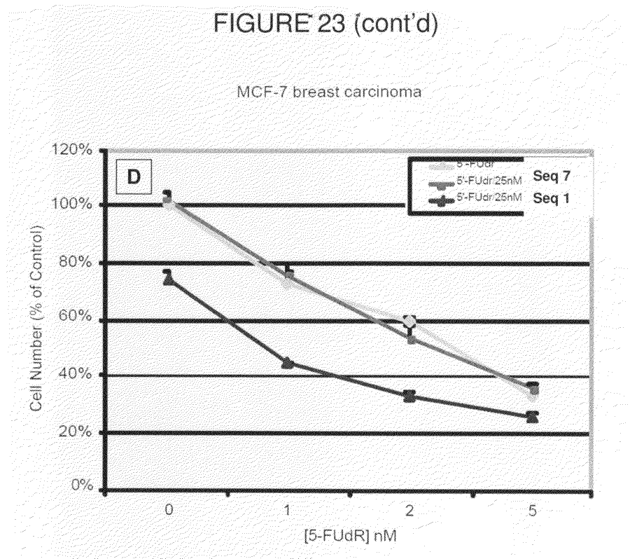

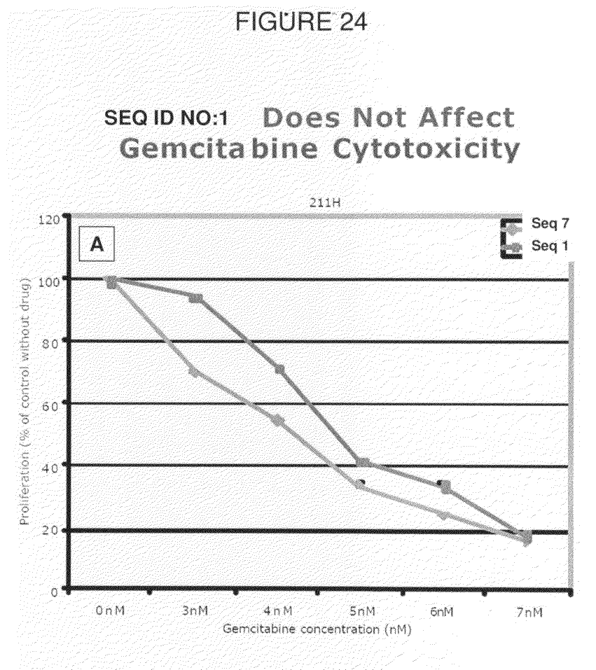

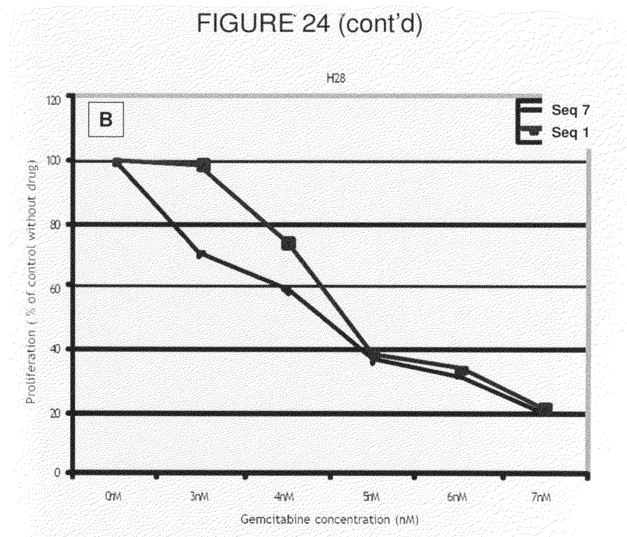

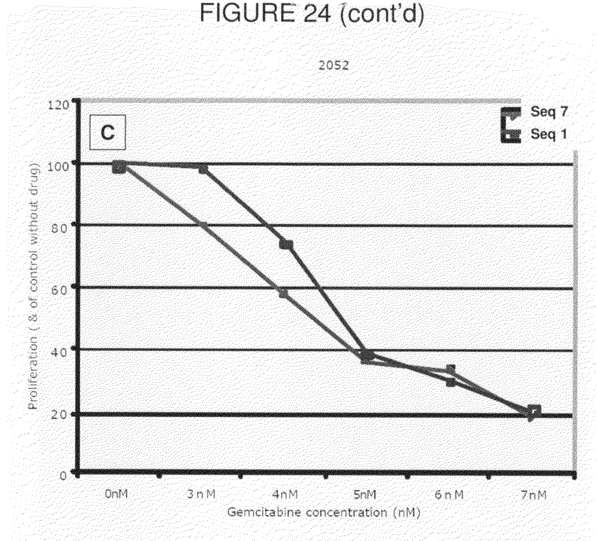

FIG. 23 illustrates that TS antisense ODN SEQ ID NO:1 also potentiates the cytotoxicity of 5-FUdR in mesothelioma cells, and to a lesser extent in MCF-7 cells. Cytotoxicity of gemcitabine in mesothelioma cells was relatively unaffected by TS antisense ODN SEQ ID NO:1 (FIG. 24).

### Example 10

### Enhancement of In Vivo Antitumour Activity of Pemetrexed by ODN 83 Against A549 Human Lung Tumours in Immunodeficient Mice

ODN 83 has been shown to reduce TS protein level and slow growth of a human tumor xenograft in nude mice when treatment was initiated immediately following injection of cells (Berg, R W, et. al., J. Pharmacol. Exp. Ther., 298, 477-484, 2001). This Example demonstrates in vivo two features of ODN 83 that will be required for its clinical usage: 1) that ODN 83 can enhance antitumor activity of a TS inhibitor in a tumor-bearing mouse; 2) that the antitumor activity of ODN 83 is successful in an established tumor.

### Materials and Methods

Cells: The human non-small cell lung cancer (NSCLC) A549 cell line was obtained from American Type Culture Collection. The cell line was tested (independent laboratory) and confirmed to be free of contaminating organisms.

Mice: Immunodeficient (nude) mice (CRL:NU-Foxn/nu) were purchased from Charles River Laboratories (Montreal, PQ, Canada).

Chemicals: ODNs 83 and 32 were generously provided by ISIS Pharmaceuticals, Inc. (Dr. Nick Dean). Pemetrexed, a product of Eli Lilly and Co. was purchased from the London Regional Cancer Program pharmacy.

Other: Cell culture media, chemicals, and plasticware were purchased from commercial sources. Matrigel was obtained from VWR International (Mississauga, ON, Canada).

Tumor Chemotherapy: Rapidly proliferating A549 cells, grown in cell culture, were harvested and prepared for injection into mice to create tumor explants. Mice were mildly anaesthetized by inhalation (isoflurane), and injected subcutaneously (s.c.) in each of 2 sites in the flank region with 7.5×10⁶ cells in 100:1 matrigel (July, L V, et al., Molecular Cancer Therapeutics, 3 (3): 223-232, 2004; Nakayama, K, et al. Cancer Res, 65: 254-263, 2005). The doses of ODN 83 and pemetrexed were chosen so that they had not antitumor effect on their own. Starting on the day that tumors reached a volume of 200 mm³ (on the order of 2.5 to 3 weeks), groups of 6 mice (except where indicated) were treated according to one of the following protocols:

(a) control—saline injection only (9 mice)
(b) 3 times per week (Mon., Wed. Fri.) for 7 weeks with antisense ODN 83 at a dose of 25 mg/kg
(c) 3 times per week (Mon., Wed., Fri.) for 7 weeks with scrambled control ODN 32 at a close of 25 mg/kg
(d) 7 weekly injections of pemetrexed at a dose of 50 mg/kg, on the day of every third injection of ODN
(e) 25 mg/kg ODN 32+50 mg/kg pemetrexed
(f) 25 mg/kg ODN 83+50 mg/kg pemetrexed

The combinations of ODNs and pemetrexed were injected according to the schedules defined in (b) through (d) (pemetrexed at time of every third ODN injection).

Once a treatment protocol was initiated on a mouse because one of its tumors was 200 mm³, the other tumor on that mouse was included in calculations if it was at least 100 mm³ at the time of the first treatment. During the entire study, mice were weighed regularly and monitored for healthy appearance and activity.

### Results

A model system was established to test ODN 83 enhancement of antitumor activity of the folate analog pemetrexed against established tumor explants of the human non-small cell lung cancer (NSCLC) cell line A549. Pemetrexed is used clinically to treat NSCLC and other tumors. Immunodeficient (nude) mice (CRL:NU-Foxn/nu) carrying established tumours were treated with: (a) the antisense ODN 83 or a scrambled control ODN 32 at 25 mg/kg intraperitoneally (IP); (b) pemetrexed at 50 or 100 mg/kg; (c) combinations of ODN (83 or 32) with 50 or 100 mg/kg pemetrexed; or (d) saline as a control. The results are shown in FIGS. 26-28.

Based on Kaplan-Meier plots of the length of time tumors took to increase their size by 100%, ODN 83 significantly enhanced the antitumor effect of 50 mg/kg pemetrexed, and that of the combined pemetrexed treatment groups (P<0.05). The median length of time for tumors to double in size was 11 to 26 days for controls, single treatments and combinations that did not include ODN 83. Median tumor size doubling time was increased to greater than 50 days when ODN 83 was added to pemetrexed. In the groups with ODN alone, 50 mg/kg pemetrexed alone, or control ODN 32 plus 50 mg/kg pemetrexed, 40-50% of tumors increased in volume by 300% or more over the 50-day period (see FIG. 29). By contrasts none of the tumors treated with ODN 83 plus pemetrexed did.

To the inventors' knowledge, this is the first demonstration in an in Vivo setting of an antisense ODN directed towards a drug target that enhanced the antitumor activity of a drug against that target.

### Summary

1) The combination of 25 mg/kg anti-TS antisense ODN 83 (targeting TS mRNA) and 50 mg/kg pemetrexed (targeting TS protein) significantly inhibited growth of established tumors of A549 human non-small cell lung carcinoma. The characteristics of tumor growth indicated anti-tumor synergy between these compounds.
2) Anti-TS antisense ODN 83 was used at a dose that, as a single agent, had no antitumor activity against established A549 tumors.
3) Pemetrexed was also used at a dose that, as a single agent, had no antitumor, activity against established A549 tumors.

Determination of whether ODN 83 abrogates the expected increase in TS protein induced by pemetrexed treatment can be assessed by conducting additional experiments as described above, in which the treated tumours are excised and changes in relative TS mRNA and protein levels measured by quantative PCR and Western blot, respectively.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

We claim:

1. A composition comprising an antisense oligonucleotide of between 10 and 100 nucleotides in length and comprising at least 10 consecutive nucleotides of a sequence according to SEQ ID NO:1 or SEQ ID NO:2 and a pharmaceutically acceptable carrier or diluent.

2. The composition according to claim 1, wherein said antisense deoxyoligonucleotide comprises the sequence according to SEQ ID NO:1 or SEQ ID NO:2.

3. The composition according to claim 1 for use in combination with a thymidylate synthase inhibitor.

4. The composition according to claim 3, wherein the thymidylate synthase inhibitor is N-(5-[N-(3,4-dihydro-2-methyl-4-oxoquinazolin-6-ylmethyl)-N-methylamino]-2-thenoyl)-L-glutamic acid.

5. The composition according to claim 3, wherein the thymidylate synthase inhibitor is pemetrexed.

6. The composition according to claim 1 for use in combination with an antiproliferative drug.

7. The composition according to claim 6, wherein the anti-proliferative drug is selected from the group consisting of: methotrexate, 5-fluorouracil, FUdR, ftorafur, capecitabine, raltitrexed and FdUR.

8. The composition according to claim 6, wherein the anti-proliferative drug is pemetrexed.

9. The composition according to claim 1, wherein the composition is in a conventional dosage form selected from the group consisting of: oral, topical, nasal, vaginal, rectal, inhalation, sub-lingual, buccal, and parenteral dosage forms.

10. The composition according to claim 1, wherein the antisense oligonucleotide is phosphorothioated, methoxy-ethoxy winged, or a combination thereof, or contains a peptide nucleic acid backbone.

11. The composition according to claim 1, wherein the antisense oligonucleotide comprises one or more locked nucleic acid nucleotides.

12. A combination product comprising an antisense oligonucleotide of between 10 and 100 nucleotides in length and comprising at least 10 consecutive nucleotides of a sequence according to SEQ ID NO:1 or SEQ ID NO:2 in combination with an anticancer agent, wherein the antisense oligonucleotide hybridizes to a 3′ untranslated region of a mammalian thymidylate synthase nucleic acid and inhibits thymidylate synthase expression in mammalian cells.

13. The combination product according to claim 12, wherein said antisense deoxyoligonucleotide comprises the sequence according to SEQ ID NO:1 or SEQ ID NO:2.

14. The combination product according to claim 12, wherein the anticancer agent is selected from the group consisting of: a thymidylate synthase inhibitor, a cytostatic agent, and an antiproliferative drug.

15. The combination product according to claim 12, wherein the anticancer agent is selected from the group consisting of N-(5-[N-(3,4-dihydro-2-methyl-4-oxoquinazolin-6-ylmethyl)-N-methylamino]-2-thenoyl)-L-glutamic acid, methotrexate, 5-fluorouracil, FUdR, ftorafur, UFT, S-1, capecitabine, and FdUR.

16. The combination product according to claim 14, wherein the anticancer agent is pemetrexed.

17. The combination product according to claim 14, wherein the antisense oligonucleotide is phosphorothioated, methoxy-ethoxy winged, or a combination thereof, or contains a peptide nucleic acid backbone.

18. The combination product according to claim 14, wherein the antisense oligonucleotide comprises one or more locked nucleic acid nucleotides.

19. A method for the treatment of cancer comprising administering to a human an effective amount of an antisense oligonucleotide of between 10 and 100 nucleotides in length and comprising at least 10 consecutive nucleotides of a sequence according to SEQ ID NO:1 or SEQ ID NO:2 and a pharmaceutically acceptable carrier or diluent.

20. The method according to claim 19, wherein said cancer is mesothelioma, breast cancer, colorectal cancer, non-small cell lung cancer, stomach cancer, oesophageal cancer, or head and neck cancer.

21. A method for the treatment of cancer comprising administering to a human an effective amount of a combination product comprising an antisense oligonucleotide of between 10 and 100 nucleotides in length and comprising at least 10 consecutive nucleotides of a sequence according to SEQ ID NO:1 or SEQ ID NO:2 in combination with an anticancer agent, wherein the antisense oligonucleotide hybridizes to a 3′ untranslated region of a mammalian thymidylate synthase nucleic acid and inhibits thymidylate synthase expression in mammalian cells.

22. The method according to claim 21, wherein said cancer is mesothelioma, breast cancer, colorectal cancer, non-small cell lung cancer, stomach cancer, oesophageal cancer, or head and neck cancer.

23. The method according to claim 21, wherein the anticancer agent is selected from the group consisting of: a thymidylate synthase inhibitor, a cytostatic agent, and an antiproliferative drug.

24. The method according to claim 21, wherein the anticancer agent is selected from the group consisting of N-(5-[N-(3,4-dihydro-2-methyl-4-oxoquinazolin-6-ylmethyl)-N-methylamino]-2-thenoyl)-L-glutamic acid, methotrexate, 5-fluorouracil, FUdR, ftorafur, UFT, S-1, capecitabine and FdUR.

25. The method according to claim 21, wherein the anticancer agent is pemetrexed.


SEQUENCE LISTING (32 sequences)

SEQ ID NO: 1 - DNA, 20 bp, Artificial Sequence
  Antisense Oligonucleotide
gccagtggcaacatccttaa

SEQ ID NO: 2 - DNA, 20 bp, Artificial Sequence
  Antisense Oligonucleotide
aagcaccctaaacagccatt

SEQ ID NO: 3 - DNA, 20 bp, Artificial Sequence
  Antisense Oligonucleotide
gcagctccgagccggccaca

SEQ ID NO: 4 - DNA, 20 bp, Artificial Sequence
  Antisense Oligonucleotide
gccggccacaggcatggcgc

SEQ ID NO: 5 - DNA, 20 bp, Artificial Sequence
  Antisense Oligonucleotide
ggcatggcgcggcgggcggg

SEQ ID NO: 6 - DNA, 19 bp, Artificial Sequence
  Antisense Oligonucleotide
ctcagctccctcagatttg

SEQ ID NO: 7 - DNA, 20 bp, Artificial Sequence
  Antisense Oligonucleotide
atgcgccaacggttcctaaa

SEQ ID NO: 8 - DNA, 1536 bp, Homo Sapien
ggggggggggggaccacttggcctgcctccgtcccgccgcgccacttggcctgcctccgtcccgccgcgccacttcgcctgcctccgtcccccgcccgccgcgccatgcctgtggccggctcggagctgccgcgccggcccttgccccccgccgcacaggagcgggacgccgagccgcgtccgccgcacggggagctgcagtacctggggcagatccaacacatcctccgctgcggcgtcaggaaggacgaccgcacgggcaccggcaccctgtcggtattcggcatgcaggcgcgctacagcctgagagatgaattccctctgctgacaaccaaacgtgtgttctggaagggtgttttggaggagttgctgtggtttatcaagggatccacaaatgctaaagagctgtcttccaagggagtgaaaatctgggatgccaatggatcccgagactttttggacagcctgggattctccaccagagaagaaggggacttgggcccagtttatggcttccagtggaggcattttggggcagaatacagagatatggaatcagattattcaggacagggagttgaccaactgcaaagagtgattgacaccatcaaaaccaaccctgacgacagaagaatcatcatgtgcgcttggaatccaagagatcttcctctgatggcgctgcctccatgccatgccctctgccagttctatgtggtgaacagtgagctgtcctgccagctgtaccagagatcgggagacatgggcctcggtgtgcctttcaacatcgccagctacgccctgctcacgtacatgattgcgcacatcacgggcctgaagccaggtgactttatacacactttgggagatgcacatatttacctgaatcacatcgagccactgaaaattcagcttcagcgagaacccagacctttcccaaagctcaggattcttcgaaaagttgagaaaattgatgacttcaaagctgaagactttcagattgaagggtacaatccgcatccaactattaaaatggaaatggctgtttagggtgctttcaaaggagcttgaaggatattgtcagtctttaggggttgggctggatgccgaggtaaaagttctttttgctctaaaagaaaaaggaactaggtcaaaaatctgtccgtgacctatcagttattaatttttaaggatgttgccactggcaaatgtaactgtgccagttctttccataataaaaggctttgagttaactcactgagggtatctgacaatgctgaggttatgaacaaagtgaggagaatgaaatgtatgtgctcttagcaaaaacatgtatgtgcatttcaatcccacgtacttataaagaaggttggtgaatttcacaagctatttttggaatatttttagaatattttaagaatttcacaagctattccctcaaatctgagggagctgagtaacaccatcgatcatgatgtagagtgtggttatgaactttatagttgttttatatgttgctataataaagaagtgttctgc

SEQ ID NO: 9 - DNA, 20 bp, Artificial Sequence
  Target sequence
ttaaggatgttgccactggc

SEQ ID NO: 10 - DNA, 20 bp, Artificial Sequence
  Target sequence
aatggctgtttagggtgctt

SEQ ID NO: 11 - DNA, 20 bp, Artificial Sequence
  Target sequence
tgtggccggctcggagctgc

SEQ ID NO: 12 - DNA, 20 bp, Artificial Sequence
  Target sequence
gcgccatgcctgtggccggc

SEQ ID NO: 13 - DNA, 20 bp, Artificial Sequence
  Target sequence
gcgccatgcctgtggccggc

SEQ ID NO: 14 - DNA, 20 bp, Artificial Sequence
  Target sequence
cccgcccgccgcgccatgcc

SEQ ID NO: 15 - DNA, 19 bp, Artificial Sequence
  Target sequence
caaatctgagggagctgag

SEQ ID NO: 16 - DNA, 30 bp, Artificial Sequence
  Target sequence
uguggccggcucggagcugccgcgccggcc

SEQ ID NO: 17 - DNA, 30 bp, Artificial Sequence
  Target sequence
gcuacagccugagagaugaauucccucugc

SEQ ID NO: 18 - DNA, 20 bp, Artificial Sequence
  Antisense Oligonucleotide
ttggatgcggattgtaccct

SEQ ID NO: 19 - DNA, 20 bp, Artificial Sequence
  Antisense Oligonucleotide
actcagctccctcagatttg

SEQ ID NO: 20 - DNA, 20 bp, Artificial Sequence
  Antisense Oligonucleotide
ccagcccaacccctaaagac

SEQ ID NO: 21 - DNA, 20 bp, Artificial Sequence
  Antisense Oligonucleotide
ggcatcccagattttcactc

SEQ ID NO: 22 - DNA, 20 bp, Artificial Sequence
  Antisense Oligonucleotide
agcatttgtggatcccttga

SEQ ID NO: 23 - DNA, 36 bp, Artificial Sequence
  oligonucleotide probe
ctagatgtggccggctcggagctgccgcgccggcca

SEQ ID NO: 24 - DNA, 36 bp, Artificial Sequence
  oligonucleotide probe
agcttggccggcgcggcagctccgagccggccacat

SEQ ID NO: 25 - DNA, 36 bp, Artificial Sequence
  oligonucleotide probe
ctagagctacagcctgagagatgaattccctctgca

SEQ ID NO: 26 - DNA, 36 bp, Artificial Sequence
  oligonucleotide probe
agcttgcagagggaattcatctctcaggctgtagct

SEQ ID NO: 27 - DNA, 20 bp, Artificial Sequence
  oligonucleotide probe
ctcttcagcacgccatggat

SEQ ID NO: 28 - DNA, 20 bp, Artificial Sequence
  oligonucleotide probe
agggtctacctttcttgcgc

SEQ ID NO: 29 - DNA, 20 bp, Artificial Sequence
  Primer
cacactttgggagatgcaca

SEQ ID NO: 30 - DNA, 25 bp, Artificial Sequence
  Primer
ctttgaaagcaccctaaacagccat

SEQ ID NO: 31 - DNA, 20 bp, Artificial Sequence
  Primer
tattgggcgcctggtcacca

SEQ ID NO: 32 - DNA, 20 bp, Artificial Sequence
  Primer
ccaccttcttgatgtcatca